(19) United States
(12) Patent Application Publication
(10) Pub. No.: US 2025/0152661 A1
(43) Pub. Date: May 15, 2025

(54) NOVEL COMBINATION TREATMENT REGIMEN OF BACTERIAL INFECTIONS

(71) Applicant: BONE SUPPORT AB, Lund (SE)

(72) Inventor: Lars LIDGREN, Tollarp (SE)

(21) Appl. No.: 18/840,256

(22) PCT Filed: Mar. 3, 2023
(86) PCT No.: PCT/EP2023/055402

§ 371 Date: Aug. 21, 2024

(30) Foreign Application Priority Data
Mar. 4, 2022 (EP) 22160214.7

(51) Int. Cl. A61K 38/14; A61K 31/496; A61K 31/7036; A61K 47/02; A61P 31/06

(52) U.S. Cl. CPC A61K 38/14; A61K 31/496; A61K 31/7036; A61K 47/02; A61P 31/06

(57) ABSTRACT

Present invention relates to a novel composition and treatment regimen in treatment of bacterial infections.

### FIELD OF THE INVENTION

Present invention relates to an innovative method of administering one or more pharmaceutical compounds to a subject in need thereof. The invention relates to the use of a mono or biphasic material, such as e.g. a ceramic material in combination with administration of one or more pharmaceutical compounds, such as e.g. two or more pharmaceutical compounds. Specifically, the invention relates to treatment of bacterial infections enabling prevention of development of bacterial resistance towards administered pharmaceutical compounds. Also, present invention relates to treatment of i.a. bacterial infections in bone, such as e.g. deep bone infections.

### BACKGROUND OF THE INVENTION

Deep bone infections (DBI's) are one of the most serious complications in orthopedic surgery. In 2018, at International consensus meeting on musculoskeletal infections, the incidences of infection covering all orthopedic subspecialties reported to range from 0.1% to 30%, at a cost of $17 000-$150000 per patient. Due to an epidemic of road traffic accidents, and the high demand for joint replacement, the incidence of DBI's with high patient morbidity and societal burden will obviously increase.

In the poorly vascularized infected bone tissue, achieving an effective and adequate sustained local concentration of a systemically given antibiotics is a challenge. The current treatment methods like long-term systemic antibiotics following local debridement may also lead to serious toxicities and the selection of antibiotic-resistant bacteria. In addition, bacteria are being sheltered residing within biofilms which further limit treatment efficacy.

In order to achieve high local antibiotics levels at minimum inhibitory concentration (MIC) or minimum biofilm eradication concentration (MBEC), surgeons in the last few decades have used polymethylmethacrylate (PMMA) containing antibiotics for local delivery. However, PMMA as a carrier does not give sustained release and lacks bone regeneration properties. There are recently approved biphasic ceramic carriers achieving high local release of gentamicin or vancomycin reporting good outcome in successful debrided bone infections. No osteoconductive biomaterial however currently contains antibiotics like tetracyclines, and rifampicin considered as cornerstone second defence antibiotics in the treatment of severe longstanding DBI's. Moreover, due to incomplete setting and mechanical properties, PMMA is not suitable as carrier for local delivery of RIF. Commercially available, bi-phasic, bioresorbable Calcium sulphate/hydroxyapatite (CaS/HA) bone void fillers, composed of 60 wt % α-calcium sulfate hemihydrate (CaS) and 40 wt % hydroxyapatite (HA) has ability to deliver biologically active molecules, hormones and anti-cancer drugs. Also, various clinical studies have reported their potential as a carrier for antibiotics such as gentamicin (GEN) and vancomycin (VAN) for the treatment of bone infections, with less side effects as compared to the commonly used systemic treatment mode. However, RIF monotherapy is not recommended due to rapid development of bacterial resistance.

### SUMMARY OF THE INVENTION

Present invention offers an improved therapy overcoming the above mentioned problems and draw-backs.

Specifically, present invention provides for one or more of,
A reduced or altogether eliminated bacterial resistance or preventing gradual build-up of bacterial resistance against the compounds employed in treating a bacterial infection.
A reduced or altogether eliminated development of planktonic bacteria and/or bacterial biofilm formation on a surface or a void, particularly the surface of bone and/or a device implanted into bone tissue.
A local extended release of high concentration of pharmaceutical compounds thereby obtaining a highly effective treatment.
An improved treatment of bone infections and in particular deep bone infections (DBI).

Thus, present invention provides for an extended release composition for use in treatment of bacterial infections or conditions related to bacterial infections.

Specifically, present invention relates to a local extended release composition for use in treatment of bacterial infections or conditions related to bacterial infections. This may allow for a locally high concentration of the employed pharmaceutical compounds which may not be possible by e.g. systemic administration of the same pharmaceutical compounds. This is particularly advantageous when treating e.g. bacterial infections which require a certain MIC (minimum inhibitory concentration) in order to result in a successful treatment.

The materials used according to the invention may in principle be any material or combination of various materials forming different phases. One such non-limiting example is any kind of bone substitute material which may suitably be hydroxyapatite (HA). Such material may be used alone or in combination with further components such as e.g. calcium sulphate (either as a hemi-hydrate and/or di-hydrate), collagen, hyaluronic acid, alginate, cellulose, poly-lactic acid etc. i.e. materials of inorganic or organic origin or polymers of natural or synthetic origin. The important feature with the material is that it has to be finely divided into micro or nanoparticles so as to provide a very large effective surface. This may be suitable achieved by any suitable technique known in the art in order to create small particles such as e.g. grinding etc. As mentioned herein, such finely divided particles may be used without addition of further materials, or may be dispersed in a second or third material. The particles dispersed or mixed with a second or more material, wherein the second or more materials are partly or totally bioabsorbable. Consequently, the material according to the invention comprises at least one first component that is not bioresorbable, or very slowly bioresorbable. This material may optionally comprise at least one second material that is bioresorbable at a higher rate than the first material.

The invention also relates to the use of one or more pharmaceutically active compounds. The pharmaceutical compound may in principle be any suitable compound for the condition or disease to be treated. Non-limiting examples are antibiotics, anticancer drugs, or any drugs used in any bone related disease or condition.

In one aspect, the invention relates to a method of treating a subject with a bacterial infection, and/or in order to prevent the occurrence of a bacterial infection. The method may comprise administering to a subject in need thereof at least a first finely divided (particulate) material, which may optionally comprise at least a second material, wherein the first material is not bioresorbable or very slowly bioresorbable and the second material or materials are bioresorbable at a higher rate than the first material, and wherein one or more, such as two or more pharmaceutical compounds are premixed with or soaked into the first material optionally comprising the second material, prior to administration to the subject.

The method may optionally comprise allowing a certain period of time to pass in order to provide for the first particulate material to be exposed to the body and/or second material or materials to be wholly or partly absorbed by the body of the subject, where after one or more pharmaceutically active compounds are additionally administered to the subject.

In one aspect of the invention, the administration in i) may optionally comprise a third material that is non resorbable but may act as a carrier for the first material and/or the second material.

In a further aspect, the invention relates to use of at least a first finely divided (particulate) material, which may optionally comprise at least a second material, wherein the first material is not bioresorbable or very slowly bioresorable, and the second material or materials are bioresorbable at a higher rate than the first material and one or more pharmaceutically active compounds, such as two or more pharmaceutically active compounds in the treatment and/or prevention of a bacterial infection.

In yet a further aspect, present invention relates to a composition, wherein the composition comprises at least a first finely divided (particulate) material, which may optionally comprise at least a second material, wherein the first material is not bioresorable or very slowly bioresorable and the second material or materials are bioresorable at a higher rate than the first material and one or more pharmaceutically active compounds, such as e.g. two or more pharmaceutically active compounds.

Present invention also relates to a compound for use in treatment of a bacterial infection. The bacterial infection may in principle be any type of bacterial infection, such as e.g. a bacterial infection in soft tissue or bone tissue. In a particular aspect, present invention relates to treatment of bacterial infection in bone tissue, such as e.g. deep bone infection (DBI).

### FIGURES

FIG. 1 illustrates the Morphological and Physio-chemical Characterization of CaS/HA-VAN/GEN with or without RIF. (A & B) Hemispherical pellets casted using an elastic mould of 4.8 mm diameter. (C & D) SEM images showing surface characteristics and pore distribution of CaS/-VAN/GEN pellets with or without RIF.

FIG. 2 (A, B) FTIR spectra from CaS/HA-VAN, pure RIF or CaS/HA-VAN+RIF (A), and CaS/HA-GEN, pure RIF or CaS/HA-GEN+RIF (B). (C) Injectability of tested CaS/HA composites. Injectability of CaS/H-VAN/+RIF, CaS/HA-RIF composites were tested at 3, 5 and 7 min, and CaS/HA-GEN/+RIF composites tested at 4, 6 and 8 min. (D) In-vitro material degradation profile of tested CaS/HA-antibiotic composites in PBS at Days 1, 3, 7, 14, 21, 28 and 35. A student t-test was used to compare the injectability and in vitro material degradation profile of CaS/HA having VAN/GEN alone or in combination with RIF. *indicates p<0.05, **indicates p<0.01, and ***indicates p<0.001.

FIG. 3 illustrates the antibiotic release profile. (A-F) Release per day and cumulative release profile of VAN, GEN and RIF from CaS/HA-VAN/GEN with or without RIF at Days 1, 3, 7, 14, 21, 28, and 35. A student t-test was used to compare the cumulative release profile CaS/HA-VAN/GEN alone or in combination with RIF. For comparison of cumulative release profile of RIF, one-way analysis of variance (ANOVA) with Dunn's multiple comparison was used. *indicates p<0.05, **indicates p<0.01, *** and indicates p<0.001.

FIG. 4 illustrates the testing of antibacterial efficacy of CaS/HA-antibiotics pellets on planktonic bacteria by continuous Kirby-Bauer disk diffusion assay. A graph data showing the zone of inhibition (ZOI) (in mm) of CaS/HA-antibiotics pellets against (A) Staphylococcus aureus ATCC 25923, and (B) Staphylococcus aureus clinical strain P3. (C) Muller Hinton agar plates inoculated with Staphylococcus aureus ATCC 25923 and clinical strain P3 showing ZOI obtained on D-1, 14, and 28, scale bar=1.6 cm. For comparison of difference in ZOI between different groups, one-way analysis of variance (ANOVA) with Tukey's multiple comparison was used. “a” in A indicates the statistical significance (p<0.0001) between CaS/HA-VAN/GEN/RIF alone in comparison to the CaS/HA-Van+RIF/CaS/HA-GEN+RIF group. “b” in A indicates the statistical significance of (p<0.0001) between CaS/HA-VAN/GEN alone in comparison to the CaS/HA-VAN/GEN+RIF group, (p<0.001) between CaS/HA-RIF in comparison to the CaS/HA-VAN+RIF group, and (p<0.05) between CaS/HA-RIF in comparison to the CaS/HA-GEN+RIF group. “c” in A indicates the statistical significance of (p<0.0001) between CaS/HA-VAN/GEN alone in comparison to the CaS/HA-VAN/GEN+RIF group, and (p<0.05) between CaS/HA-RIF in comparison to the CaS/HA-VAN+RIF group. “a” in B indicates the statistical significance of (p<0.0001) between CaS/HA-VAN/GEN alone in comparison to the CaS/HA-VAN/GEN+RIF group, and (p<0.01) between CaS/HA-RIF in comparison to the CaS/HA-VAN+RIF group. “p” in B indicates the statistical significance of (p<0.0001) between CaS/HA-VAN/GEN alone in comparison to the CaS/HA-VAN/GEN+RIF group, (p<0.05) between CaS/HA-RIF in comparison to the CaS/HA-VAN+RIF group, and (p<0.01) between CaS/HA-RIF in comparison to the CaS/HA-GEN+RIF group. “Z” in B indicates the statistical significance of (p<0.0001) between CaS/HA-VAN/GEN alone in comparison to the CaS/HA-VAN/GEN+RIF group, and (p<0.05) between CaS/HA-RIF in comparison to the CaS/HA-VAN+RIF group. “S” in B indicates the statistical significance of (p<0.0001) between CaS/HA-VAN alone in comparison to the CaS/HA-VAN+RIF group, (p<0.001) between CaS/HA-RIF in comparison to the CaS/HA-VAN+RIF group, (p<0.01) between CaS/HA-GEN alone in comparison to the CaS/HA-GEN+RIF group, and (p<0.05) between CaS/HA-RIF in comparison to the CaS/HA-GEN+RIF group. “ε” in B indicates the statistical significance of (p<0.0001) between CaS/HA-VAN alone in comparison to the CaS/HA-VAN+RIF group, (p<0.001) between CaS/HA-GEN in comparison to the CaS/HA-GEN+RIF group. ND—Not done.

FIG. 5 illustrates the testing of antibacterial efficacy of CaS/HA-antibiotics pellets on planktonic bacteria by continuous Kirby-Bauer disk diffusion assay for 28 days. A graph data showing the zone of inhibition (ZOI) (in mm) of CaS/HA-antibiotics pellets against (A) Staphylococcus aureus ATCC 25923, and (B) Staphylococcus aureus clinical strain P-3.

FIG. 6 illustrates the antibacterial effect of pellets after day-35. (A) A graph data showing the antibacterial effect of the pellets placed in PBS for 35 days. (B) Muller Hinton agar plates inoculated with Staphylococcus aureus ATCC 25923 showing ZOI, scale bar=2.6 cm.

FIG. 7 illustrates the efficacy of antibiotic fractions from D-1, 7, 14, 21, 28 & 35 on preformed biofilms. (A-F) Graphical representation of the Staphylococcus aureus ATCC 25923 and clinical strain P3 biofilm destruction obtained by crystal violet staining. A student t-test was used to compare the differences in biofilm destruction between CaS/HA-VAN/GEN with or without RIF. *indicates p<0.05, **indicates p<0.01, ***indicates p<0.001, and ****indicates p<0.0001.

FIG. 8 illustrates the SEM images of Staphylococcus aureus ATCC 25923 biofilm destruction. For all tested CaS/HA-antibiotic groups, biofilm destruction and antibacterial effects were evident on day-7 and 14. At day-28, number of planktonic bacteria were increased and biofilm were visible. Green arrow indicates biofilm while violet arrow indicates destructed biofilm. Red arrow indicates dead bacteria. White arrow indicates membrane disruption and extravasation of cytoplasmic components of bacteria. Scale bar=1 μm.

### DETAILED DESCRIPTION OF THE INVENTION

Present invention relates to the use of one or more materials. The material may be mono-phasic or comprising different phases, such as e.g. bi-phasic, tri-phasic, tetra-phasic, penta-phasic etc. In the context of the invention the material has to have at least one component that is not bioresorbable or very slowly bioresorbable.

By the term “bioresordable” or bioresorbable” is meant a material that can be broken down and absorbed by the body, and thus does not need to be removed manually. Such material may e.g. be integrated by the body, such as e.g. be integrated into bone tissue, or otherwise metabolized by the organism.

One non-limiting example of a bioresorbable material is apatite or hydroxyapatite (HA) in any configuration or crystal form, or any suitable bone substitute material (alloplasts or calcium phosphates) or bioglass. These materials are slowly bioresorbable and may eventually be integrated into the bone tissue of the subject. Other examples are autologous bone grafts (autograft) or cancellous bone. Moreover, various other bone grafts may be used such as e.g. isografts, allografts, xenografts etc. These materials may have a wholly or partially crystalline microstructure. Another feature is that these materials according to the invention are finely divided into small particles such as e.g. micro or even nanoparticles. As a consequence thereof, the effective surface of a certain amount of such material is very large. It is to be noted that according to the invention the above described material may be denoted as a first material. The first material may comprise several different components such that e.g. HA (hydroxyapatite) is mixed with e.g. cancellous bone or any other suitable bone substitute material. Merely for sake of completion, hydroxyapatite which is also referred to as hydroxylapatite, has the formula Ca₅(PO₄)₃(OH), but may also be written as Ca₁₀(PO₄)₆(OH)₂ to denote that the crystal unit cell comprises two entities. This entity may be denoted or abbreviated as “HA” in present text.

In a particular embodiment, the first material is hydroxyapatite (HA) in any configuration or particle size.

As mentioned above the first material is absorbed very slowly by the organism. Typically, the period of absorption may be in the range of weeks, months and even years, such as e.g. about 2 weeks, such as e.g. about 3 weeks, such as e.g. about 4 weeks, such as e.g. about 5 weeks, such as e.g. about 6 weeks, such as e.g. about 7 weeks, such as e.g. about 2 months, such as e.g. about 3 months, such as e.g. about 4 months, such as e.g. about 6 months, such as e.g. about 8 months, such as e.g. about 10 months, such as e.g. about 12 months, such as e.g. about 18 months, such as e.g. about 2 years, or such as e.g. several years etc. after administration to the subject. In one aspect, the first material may not be absorbed at all by the organism.

In one aspect, the first material is absorbed very slowly by the organism, such as e.g. about 6 months, such as e.g. about 8 months, such as e.g. about 10 months, such as e.g. about 12 months, such as e.g. about 18 months, such as e.g. about 2 years, or between about 6 months to about 2 years after administration to the subject. In a further aspect the first material is absorbed very slowly by the organism, such as e.g. about 6 months to about 2 years.

The first material may optionally be mixed with a second material. The second material may comprise one component or may be mixtures of several components. The relation between the first material and the second material is such that the second material is absorbed by the body of the subject at a higher rate than the first material. Non-limiting examples of a second material is one or more of fibrogen, dextran, hyaluronic acid, alfatachycalciferol, calcium sulphate (hemi-hydrate and/or di-hydrate), collagen, various forms of cellulose such as carboxymethyl cellulose (CMC) or sodium-CMC etc or other polysaccharides such as e.g. agarose. Other non-limiting examples are blood or blood derived components such as a fibrin clot.

In one aspect, the second material may comprise e.g. calcium sulphate (hemi-hydrate and/or di-hydrate) in any crystal configuration. Calcium sulphate may be abbreviated or denoted as “CaS” in present text.

Such materials, referred herein as a “second material”, are usually absorbed by the body within a few days, week or months, such as e.g. about 1 day, such as e.g. about 2 days, such as e.g. about 3 days, such as e.g. about 4 days, such as e.g. about 5 days, such as e.g. about 6 days, such as e.g. about 7 days, such as e.g. about 8 days, such as e.g. about 9 days, such as e.g. about 10 days, such as e.g. about 11 days, such as e.g. about 12 days, such as e.g. about 13 days, such as e.g. about 2 weeks, such as e.g. about 3 weeks, such as e.g. about 4 weeks, such as e.g. about 5 weeks, such as e.g. about 6 weeks, such as e.g. about 7 weeks, such as e.g. about 2 months, such as e.g. about 3 months, or such as e.g. about 4 months etc.

In one aspect, the second material is usually absorbed by the body of the subject within e.g. about 1 day, such as e.g. about 2 days, such as e.g. about 3 days, such as e.g. about 4 days, such as e.g. about 5 days, such as e.g. about 6 days, such as e.g. about 7 days, such as e.g. about 8 days, such as e.g. about 9 days, such as e.g. about 10 days, such as e.g. about 11 days, such as e.g. about 12 days, such as e.g. about 13 days, such as e.g. about 2 weeks, such as e.g. about 3 weeks, such as e.g. about 4 weeks, such as e.g. about 5 weeks, such as e.g. about 6 weeks after administration to the subject.

In a further aspect, the second material is usually absorbed by the body of the subject within e.g. about 1 day to about 6 weeks after administration to the subject. In one aspect, the second material is usually absorbed by the body of the subject within about 6 weeks after administration to the subject.

In yet a further aspect, when the composition comprises a first and a second material, the first material is absorbed very slowly by the organism, such as e.g. in the range of weeks, months and even years, such as e.g. about 2 weeks, such as e.g. about 3 weeks, such as e.g. about 4 weeks, such as e.g. about 5 weeks, such as e.g. about 6 weeks, such as e.g. about 7 weeks, such as e.g. about 2 months, such as e.g. about 3 months, such as e.g. about 4 months, such as e.g. about 6 months, such as e.g. about 8 months, such as e.g. about 10 months, such as e.g. about 12 months, such as e.g. about 18 months, such as e.g. about 2 years, or such as e.g. several years etc., and wherein the second material is usually absorbed by the body within a few days, week or months, such as e.g. about 1 day, such as e.g. about 2 days, such as e.g. about 3 days, such as e.g. about 4 days, such as e.g. about 5 days, such as e.g. about 6 days, such as e.g. about 7 days, such as e.g. about 8 days, such as e.g. about 9 days, such as e.g. about 10 days, such as e.g. about 11 days, such as e.g. about 12 days, such as e.g. about 13 days, such as e.g. about 2 weeks, such as e.g. about 3 weeks, such as e.g. about 4 weeks, such as e.g. about 5 weeks, such as e.g. about 6 weeks, such as e.g. about 7 weeks, such as e.g. about 2 months, such as e.g. about 3 months, or such as e.g. about 4 months etc.

In a further aspect, when the composition comprises a first and second material, the first material is absorbed very slowly by the organism, such as e.g. about 6 months, such as e.g. about 8 months, such as e.g. about 10 months, such as e.g. about 12 months, such as e.g. about 18 months, such as e.g. about 2 years, or between about 6 months to about 2 years after administration to the subject, and wherein the second material is usually absorbed by the body of the subject within e.g. about 1 day, such as e.g. about 2 days, such as e.g. about 3 days, such as e.g. about 4 days, such as e.g. about 5 days, such as e.g. about 6 days, such as e.g. about 7 days, such as e.g. about 8 days, such as e.g. about 9 days, such as e.g. about 10 days, such as e.g. about 11 days, such as e.g. about 12 days, such as e.g. about 13 days, such as e.g. about 2 weeks, such as e.g. about 3 weeks, such as e.g. about 4 weeks, such as e.g. about 5 weeks, such as e.g. about 6 weeks, or within e.g. about 1 day to about 6 weeks after administration to the subject.

The material, being either a mono-phasic material or mixtures of several phases, may be formulated in any form, such as e.g. a liquid such as e.g. a suspension or solution, paste form, semi-solid, or a solid formulation. The first material may be dispersed in the second material, such as e.g. HA (hydroxyapatite) being dispersed or mixed in a paste of calcium sulphate (CaS), and may be regarded as embedded in CaS. Solid formulations may be in any form such as e.g. tablets, pellets, beads, sticks etc. of any suitable size such as allowing implantation into the body of the subject or injection into the body of the subject. With respect to liquid or semi-solid compositions, these may be administered to the subject by means of injection or any other suitable means. The composition comprising the first and optionally a second material may be such that once administered to the subject, the composition cures into a solid at the site of administration. The administration of the composition may suitably be at the site of the body in need of treatment such as e.g. into or near bone tissue either for treatment of an ongoing bacterial infection or for prophylactic treatment or preventive treatment of bacterial infections.

In one aspect, the first material may be hydroxyapatite (HA) in any form and the second material may be calcium sulphate (CaS) in any form or crystal configuration. In a further aspect, the particulate first material (such as e.g. HA) may be embedded into the second material (such as e.g. CaS or calcium phosphate).

As is apparent from the description, the first and second material may act as carriers for the one or more pharmaceutical compounds and are from a pharmacological perspective inert.

In a further aspect, the composition according to the invention may comprise only a first material and one or more pharmaceutical compounds, i.e. without the presence of a second material.

The material or composition according to the invention may optionally further comprises any suitable pharmaceutical compound, such that the material or composition according to the invention which is administered to the subject comprises a first material, and optionally a second material, and a pharmaceutically active compound.

Specifically, the pharmaceutically active compound, which may be one or more pharmaceutically active compounds, may be any compound capable of treating a bacterial infection. It is to be understood that such compound may be further combined with other classes of pharmaceutical compounds such as e.g. bisphosphonates or drugs capable of treating cancer.

It is also to be noted that the material or composition according to the invention may also be applied or adhered at least partly to any surface. The surface may be the surface of any object that is to be implanted into the body of the subject, such as e.g. hip or joint replacement etc., or may be deposited into e.g. the surface of a membrane, disc or pin to be implanted into the body of the subject. Other examples may be other devices or objects such as e.g. catheters, spinal cage, any grid or net-like construction, tubes of any kind, stents etc. that are implanted or inserted into the body for a limited or unlimited period of time. With respect to devices, any kind of prosthetics and implants may be envisaged. In one non-limiting example, such device may be e.g. an object in any form of metal, alloy, polymer, elastomers etc. suitable for implanting into the body of a subject in need. Suitably, the device may at least partly be coated with a composition according to the invention

As mentioned herein, the composition according to the invention may be administered into any site of the body of the subject. Specific tissue may be any soft tissue or may be bone tissue. As the composition may be administered to any part of the body, present invention also relates to and enables an in situ generated medical device. This may be achieved by administering systemically and/or locally of one or more pharmaceutical compositions to a targeted device thereby changing the device and to locally actively combat for instance infections.

In the context of present invention, the methods and compositions and use of the compositions as disclosed herein may be for use in the treatment of bacterial infections of any kind. In a specific embodiment, present invention relates to treating bacterial infections in bone tissue.

In a further specific embodiment, present invention relates to treatment of deep bone infections (DBI's).

In one aspect, the pharmaceutical composition being administered to the subject may be a pharmaceutical compound displaying high affinity to the first material. Such pharmaceutical composition may be further conjugated or derivatised with a further pharmaceutical compound that may have no or very low affinity for the first material. Such further pharmaceutical composition may be covalently or indirectly bound or complexed to the pharmaceutical compound displaying high affinity to the first material. In one instance, the first material may be HA (hydroxyapatite). A suitable pharmaceutical compound having high affinity for HA may be, but not limited to, bisphosphonates. In this non-limiting case, the bisphosphonate may be further conjugated to or derivatised with e.g. an antibiotic or compound capable of treating a bacterial infection.

Once the material or composition according to the invention has been administered to the subject, the second material (in case it is present) is absorbed by the body or erodes in the body of the subject so as to expose the finely particulate structure or the first material having a very high effective surface area. The period during which the absorption or erosion of the second material takes place varies with the material used as second material. Thus the period in questions may be at least a few hours, or within e.g. about 1 day, such as e.g. about 2 days, such as e.g. about 3 days, such as e.g. about 4 days, such as e.g. about 5 days, such as e.g. about 6 days, such as e.g. about 7 days, such as e.g. about 8 days, such as e.g. about 9 days, such as e.g. about 10 days, such as e.g. about 11 days, such as e.g. about 12 days, such as e.g. about 13 days, such as e.g. about 2 weeks, such as e.g. about 3 weeks, such as e.g. about 4 weeks, such as e.g. about 5 weeks, such as e.g. about 6 weeks, or within e.g. about 1 day to about 6 weeks after administration to the subject. After the absorption period of the second material, one or more further pharmaceutical compositions may be optionally and additionally administered to the subject. Consequently, one or more pharmaceutical compositions may be administered after at least a few hours, or within e.g. about 1 day, such as e.g. about 2 days, such as e.g. about 3 days, such as e.g. about 4 days, such as e.g. about 5 days, such as e.g. about 6 days, such as e.g. about 7 days, such as e.g. about 8 days, such as e.g. about 9 days, such as e.g. about 10 days, such as e.g. about 11 days, such as e.g. about 12 days, such as e.g. about 13 days, such as e.g. about 2 weeks, such as e.g. about 3 weeks, such as e.g. about 4 weeks, such as e.g. about 5 weeks, such as e.g. about 6 weeks, or within e.g. about 1 day to about 6 weeks after the first and/or second material has been administered to the subject.

Alternatively, the one or more pharmaceutical compositions are administered e.g. about 6 weeks to about 1 year, about 18 months, or about 2 years after administration of the composition according to the invention.

The one or more pharmaceutical compositions may be administered one or more times, such as e.g. 2 or more time, 3 or more times, 4 or more times, 5 or more times, 6 or more times, 7 or more times, 8 or more times, 9 or more times, 10 or more times etc.

In another aspect, present invention may comprise a methodology wherein a first, optionally a second material further comprising one or more pharmaceutical compounds is/are administered to a subject, where after no further administrations of pharmaceutical compounds takes place.

As is implied in present description, the composition according to the invention may be an extended release composition, wherein the one or more pharmaceutical compounds are released from the first and optionally the second material over an extended period of time. In a particular aspect, the composition is administered to a particular tissue, whereby the one or more pharmaceutical compounds are released from the first and optionally the second material over an extended period of time at or near the site of administration. This in turn may result in not having to administer the same or different pharmaceutical compounds at any later time.

The composition according to the invention enables the one or more pharmaceutical compounds to be released from the first and optionally second material for an extended period of time such as e.g. at least about 1 day, at least about 3 days, at least about 7 days, at least about 14 days, at least about 21 days, at least about 28 days, or at least about 35 days or more.

The one or more pharmaceutical compositions may comprise two or more different active ingredients, such as 3 or more, 4 or more, or 5 or more etc. Such active ingredients may display different affinities toward the first and the second material.

In the regimen relating to administration of one or more pharmaceutical compositions, the pharmaceutical composition may comprise one or more same or different active ingredients at the first instance of administration, whereas upon a second administration, the pharmaceutical composition may comprise one or more same or different active ingredients, which are optionally different from the ones administered at the first instance or occasion of administration of a pharmaceutical composition.

In a one aspect, the first and optionally the second material may be pre-soaked with one or more pharmaceutical compounds. Alternatively, the first and optionally the second material, may be mixed together with one or more pharmaceutical compounds. The pre-soaking or mixing may take place prior to any administration to the subject.

In another aspect, the pharmaceutical compound may be administered to the subject after administration of the first and optionally the second material.

In yet a further aspect, the pharmaceutical compound may be pre-soaked or mixed with the first and optionally the second material, and may optionally also be administered one or more times after administration of the pre-soaked or mixed first and optionally the second material.

The pre-soaking or mixing may be such that one type of one or more pharmaceutical compounds is/are pre-soaked into or mixed with a first material, and the same or a different type of one or more pharmaceutical compounds is/are pre-soaked into or mixed with a second material.

The one or more pharmaceutical compounds according to the invention may in principle be any pharmaceutical compound that shows an affinity for the first material used in the composition according to the invention. In one aspect, the pharmaceutical compound may be any pharmaceutical compound showing an affinity for hydroxyapatite particles.

As mentioned above, the treatment may comprise the use of one or more same or different classes of pharmaceutical compounds. These may either be administered concurrently or sequentially in any order as needed or deemed necessary.

Present invention also relates to a composition, wherein the one or more administered drugs display different affinities towards the first and the second material. Thus in one aspect, present invention relates to a bi-phasic or multi-phasic composition, various different pharmaceutical compounds may be employed such that e.g. one pharmaceutical compound shows high affinity towards the particulate first material (and low affinity towards the second material), and another pharmaceutical compound shows high affinity towards the second material (and low affinity towards the first material) etc. In one aspect, the second material may be mixed or pre-loaded with a pharmaceutical compound which optionally shows high affinity for the second material, and then further mixed with the first material. Also, alternatively, the first material may be mixed or exposed to a pharmaceutical compound displaying high affinity to the first material. The above mentioned first and second materials may be mixed and administered to the subject in need. This allows for a method or treatment regimen where one pharmaceutical compound very slowly is allowed to be released from the composition according to the invention (the first material), whereas one pharmaceutical compound is released relatively quickly from the second material which is eroded relatively quickly be the body of the subject in question.

In one aspect, present invention also relates to a composition comprising a first particulate material, wherein the material is present in nano-sized particles. The material may or may not be pre-loaded with a pharmaceutical compound. Such particles may be administered as a suspension or otherwise dispersed in any suitable solvent such as e.g. as saline solution, which is suitably a physiological saline solution. Such composition may be used for various applications such as e.g. administration into or near an infected tissue, where the particles will penetrate the bacterial cells to exert their action.

The one or more pharmaceutical compound may be any antibiotic compound such as e.g. rifampicin or any ansamycin such as e.g. geldanamycin, herbimycin A, macbecin, natalamycin, streptovaricin or rifamycins in general or any derivatives thereof. The antibiotic compound may also be a daptomycin.

The antibiotic may be any type of fluoroquinolone, such as e.g. flumequine, oxolinic acid, rosoxacin, ciprofloxacin, fleroxacin, lomefloxacin, nadifloxacin, norfloxacin, ofloxacin, pefloxacin, rufloxacin, balofloxacin, levofloxacin, trovafloxacin, grepafloxacin, levofloxacin, pazufloxacin, sparfloxacin, temafloxacin, clinafloxacin, gatifloxacin, moxifloxacin, sitafloxacin, prulifloxacin, besifloxacin, delafloxacin, ozenoxacin, danofloxacin, difloxacin, enrofloxacin, ibafloxacin, marbofloxacin, orbifloxacin, and sarafloxacin or nalidixic acid, or any combinations thereof.

In one aspect the antibiotic may be any type of β-lactam, such as e.g. penicillin, benzathine penicillin, benzylpenicillin, phenoxymethylpenicillin, procaine penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, piperacillin, cefazolin, cephalexin, cephalosporin C, cephalothin, cefaclor, cefamandole, cefuroxime, cefotetan, cefoxitin, cefixime, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, aztreonam, tigemonam, nocardicin A, tabtoxinine β-lactam, clavulanic acid, tazobactam, sulbactam, avibactam, or any combinations thereof.

In one aspect the antibiotic may be any type of glycopeptide such as e.g. vancomycin, teicoplanin, or ramoplanin, or any combinations thereof.

In a further aspect, the antibiotic may be a lipopeptide such as e.g. bacillomycins, daptomycin, echinocandins (e.g. caspofungin), iturin A, mycosubtilin, surfactin, or any combinations thereof.

In yet a further aspect the antibiotic may be an aminoglycoside such as e.g. amikacin, apramycin, arbekacin, astromicin, bekanamycin, dibekacin, dihydrostreptomycin, elsamitrucin, fosfomycin/tobramycin, gentamicin, hygromycin B, isepamicin, kanamycin A, kasugamycin, legonmycin, lividomycin, micronomicin, neamine, neomycin, netilmicin, nourseothricin, paromomycin, plazomicin, ribostamycin, sisomicin, streptoduocin, streptomycin, tobramycin, totomycin, and verdamicin, or any combinations thereof.

In one aspect, the antibiotic may be a tetracycline of any kind, such as e.g. tetracycline, chlortetracycline, oxytetracycline, demeclocycline, iymecycline, meclocycline, methacycline, minocycline, rolitetracycline, tigecycline, omadacycline, sarecyclin or any combinations thereof.

In one aspect, the antibiotic may be a macrolide, such as e.g. azithromycin, clarithromycin, erythromycin, fidaxomicin, telithromycin or any combinations thereof.

In one aspect, the antibiotic may be a any type of oxazolidinone such as e.g. eperezolid, linezolid, posizolid, radezolid, ranbezolid, sutezolid, tedizolid or any combinations thereof.

The pharmaceutical compound may also be a bisphosphonate such as e.g. zoledronic acid, etidronic acid, clodronic acid, tiludronic acid, pamidronic acid, neridronic acid, olpadronic acid, alendronic acid, ibandronic acid, risendronic acid or any suitable slats thereof. The bisphosphonate be also be a salt wherein a radioactive compound is present or a radioactive compound as ⁹⁹Tc or ²²³Ra or strontium or samarium.

Other agents may be employed are e.g. compounds that are X-ray visible or detectably by standard means such as e.g. fluorescence or MR or compounds used in PET-CT. One example is e.g. compounds comprising ¹⁸F such as e.g. Na¹⁸F or fluorodeoxyglucose (¹⁸F).

As mentioned herein, present invention relates to pharmaceutical compounds, wherein said compounds may have different affinities towards the first material which may be, but not limited to, e.g. hydroxyapatite in any form. Thus, present invention relates to pharmaceutical compounds with high affinity towards the first material and/or pharmaceutical compounds with low or no affinity towards the first material.

In one non-limiting example, pharmaceutical compounds with high affinity towards the first material may be e.g. one or more bisphosphonates, tetracyclines, anthracyclines, ansamycins or the likes.

In one aspect, the tetracyclines which display high affinity towards the first material may comprise tetracycline, Chlortetracycline, Oxytetracycline, Demeclocycline, Lymecycline, Meclocycline, Methacycline, Minocycline, Rolitetracycline, Doxycycline, Tigecycline, Eravacycline, Sarecycline, or Omadacycline, or any mixtures thereof.

In yet a further aspect, the pharmaceutical compound displaying a high affinity towards the first material may be tetracycline.

In one aspect, pharmaceutical compounds which display high affinity towards the first material may comprise, may be one or more ansamycins, and may comprise rifampicin, naphthomycins, geldanamycin, or ansamitocin or any combinations thereof.

In yet a further aspect, the pharmaceutical compound displaying a high affinity towards the first material may be rifampicin.

In one aspect, pharmaceutical compound displaying a high affinity towards the first material may be one or more anthracyclines, which may be e.g. doxorubicin, daunorubicin, epirubicin and idarubicin, or any combinations thereof.

Also, present invention relates to one or more pharmaceutical compound displaying a low or no affinity towards the first material.

In one aspect, the pharmaceutical compound displaying a low or no affinity towards the first material may be one or more compounds of the aminoglycoside class of antibiotics, and may be e.g. gentamicin, tobramycin, amikacin, plazomicin, streptomycin, neomycin, and paromomycin, or any combinations thereof.

In one aspect, the aminoglycoside antibiotic may be gentamycin.

In yet a further aspect, the pharmaceutical compound displaying a low or no affinity towards the first material may be one or more glycopeptide antibiotics. Such glycopeptide antibiotics may be e.g. the anti-infective antibiotics vancomycin, teicoplanin, telavancin, ramoplanin and decaplanin, corbomycin, complestatin and the antitumor antibiotic bleomycin, or any combinations thereof.

In one particular aspect, present invention relates to vancomycin as the glycopeptide antibiotic.

In yet a further aspect, the pharmaceutical compounds may be a combination with one or more glycopeptide antibiotics and one or more ansamycins.

Alternatively, the pharmaceutical compounds may be a combination with one or more aminoglycoside antibiotic and one or more ansamycins.

Moreover, the pharmaceutical compounds may be a combination with one or more glycopeptide antibiotics and one or more ansamycins, and one or more aminoglycoside antibiotics.

In a further aspect, the pharmaceutical compounds may be a combination of vancomycin and rifampicin.

Alternatively, the pharmaceutical compounds may be a combination of gentamicin and rifampicin.

As yet a further alternative, the pharmaceutical compounds may be a combination of gentamicin, vancomycin and rifampicin.

Thus, as is apparent from the description, present invention relates to a combination of two or more pharmaceutical compounds, such as three or more pharmaceutical compounds, such as four or more pharmaceutical compounds etc.

According to the invention, present invention relates to a composition comprising a first and optionally also a second material. The composition further comprises one or more pharmaceutical compounds used in the treatment of a bacterial infection, i.e. compounds known for use in the treatment of bacterial infections.

In one aspect, the invention relates to a composition comprising a first and a second material, which further comprises two or more pharmaceutical compounds used in the treatment of a bacterial infection, i.e. compounds known for use in the treatment of bacterial infections.

In yet a further aspect, the invention relates to a composition comprising a first and a second material, which further comprises at least one pharmaceutical compound used in the treatment of a bacterial infection, i.e. compounds known for use in the treatment of bacterial infections. The at least one pharmaceutical compound may be selected from the group of ansamycins, glycopeptides, and aminoglycosides.

In one aspect, the invention relates to a composition comprising a first which may be e.g. hydroxyapatite (HA) and a second material which may be calcium sulphate (CaS), which further comprises at least one pharmaceutical compound used in the treatment of a bacterial infection, i.e. compounds known for use in the treatment of bacterial infections. The at least one pharmaceutical compound may be selected from the group of ansamycins, glycopeptides, and aminoglycosides. Specifically, the pharmaceutical composition according to the invention may comprise a combination of at least one ansamycin and at least one glycopeptide. In another aspect, the pharmaceutical composition according to the invention may comprise a combination of at least one ansamycin and at least one aminoglycoside.

Thus in one aspect, present invention relates to a combination of a first material and optionally a second material and a combination of vancomycin and rifampicin. The first material may be e.g. hydroxyapatite (HA). The second material which is optionally present, may be e.g. calcium sulphate (CaS) or calcium phosphate (CaP).

Alternatively, present invention relates to a first material and optionally a second material and a combination of gentamicin and rifampicin. The first material may be hydroxyapatite (HA). The second material which is optionally present, may be calcium sulphate (CaS) or calcium phosphate (CaP).

Thus in one aspect, the second material may be e.g. calcium sulphate (CaS) in any configuration or hydration.

In another aspect, the second material may be e.g. calcium phosphate (CaP) in any configuration or hydration. In this respect, calcium phosphate may not be hydroxyapatite (HA). In another aspect, calcium phosphate may be a salt comprising Ca²⁺ combined with either of PO₄⁻³, HPO₂⁻⁴, or H₂PO⁻⁴. Other non-limiting examples are monocalcium phosphate (Ca(H₂PO₄)₂ and Ca(H₂PO₄)₂(H₂O), or dicalcium phosphate (CaHPO₄(H₂O)₂ or CaHPO₄(H₂O)), or tricalcium phosphate (Ca₃(PO₄)₂), or octacalcium phosphate (Ca₈H₂(PO₄)₆·5H₂O). In another aspect, CaP may be intended to mean amorphous calcium phosphate which is a glassy precipitate of variable composition that may be present in biological systems.

In yet a further aspect, present invention relates to a first material and optionally a second material and a combination of vancomycin, gentamicin and rifampicin. The first material may be hydroxyapatite (HA). The second material which is optionally present, may be calcium sulphate (CaS) or calcium phosphate (CaP).

As mentioned herein, present invention relates to use of the composition and pharmaceutical compounds as disclosed herein in medicine. In a particular aspect, present invention relates to use of the composition and pharmaceutical compounds as disclosed herein in the treatment, or prevention, or amelioration of bacterial infections in any type of tissue.

In one particular aspect, present invention relates to a composition for use in treating, or preventing, or reducing or altogether eliminating the emergence of bacterial resistance towards any antibiotic compound. Thus, in a particular aspect, present invention relates to a composition for use in treating, or preventing, or reducing or altogether eliminating the emergence of bacterial resistance towards any antibiotic compound, such as e.g. bacterial strains exhibiting rifampicin resistance.

In another aspect, present invention relates to the composition and pharmaceutical compounds as disclosed herein for use in treatment in bacterial infections in any type of tissue, such as e.g. in bone tissue and in particular deep bone infections (DBIs).

Present invention also enables prevention, or prolongs the period for development of bacterial resistance towards the administered pharmaceutical compounds.

Present invention also enables an effective treatment of, or reduction of, or elimination of, or prevention of planktonic bacteria.

Present invention also enables an effective treatment or prevention of formation of biofilm and may also enable treatment of already formed biofilms on a tissue surface.

Present invention also relates to use of one or more pharmaceutical compounds displaying a low or no affinity towards the first material, and/or pharmaceutical compounds displaying a high affinity towards the first material and thus a combination of the two.

As is mentioned herein, the first and/or second material may be pre-soaked with one or more pharmaceutical compounds prior to implanting the first and/or second material into a subject. Alternatively, one or more pharmaceutical compounds may be administered after the first and/or second material has been implanted into a subject. In yet a further alternative, the first and/or second material may be pre-soaked with one or more pharmaceutical compounds and implanted into a subject after which additional pharmaceutical compounds are being administered to the subject. As is apparent from the invention, the pharmaceutical compounds may have different affinities towards the first material.

Thus according to the methods of present invention, areas of clinical interest may in particular be in relation to bone regeneration, or treatment of bacterial infections of any kind, such as e.g. treating bacterial infections in bone tissue.

It is to be understood that the administration of the material or compound according to the invention may be administration into any tissue such as any soft tissue, or organ, or may be administered near or into bone tissue.

With respect to administration of the pharmaceutically active compound, it is to be understood that any conventional method of administration may be employed which has been previously approved by clinical authorities. If for example a drug has been approved for oral administration, the same administration rout may be employed according to the invention. Thus, the administration is usually results in the pharmaceutically active compound entering the systemic circulation of the subject. As previously mentioned, the administration may be e.g. oral or intravenous.

In another aspect of the invention, the pharmaceutically active compound may be administered near or into e.g. an implant. In such instance, the pharmaceutically active compound may be deposited in any form of formulation and be e.g. injected near the implant or deposited into the surface of a pin, or absorbed in any suitable carrier that is inserted into cavities of the implant. The pin, may be broken off after insertion into the implant.

Overall, according to present, the first and optionally the second material which may further have been presoaked or mixed with one or more pharmaceutical compounds, may be administered to a subject in need thereof in any suitable way. This may include e.g. administration by injection. In this respect, the injection may be in form of a paste comprising the first and optionally the second material which may have been further premixed or soaked with one or more pharmaceutical compounds, wherein the paste is allowed to cure in the tissue. Alternatively, the curing may take place prior to administering of the composition as a whole to the subject. As mentioned herein, the one or more pharmaceutical compounds may be further administered after administration of the first and optionally the second material (which may or may not have been premixed or soaked with one or more pharmaceutical compounds).

As mentioned herein the first material used according to the invention has to be in a finely divided solid state, such as e.g. in small particles so as to provide for a high effective surface. The first material may be such that 1 ml of the material contains at least 0.5 million particles, such as e.g. at least 1 million particles, such as e.g. at least 2 million particles, such as e.g. at least 3 million particles, such as e.g. at least 4 million particles, such as e.g. at least 5 million particles, or such as e.g. at least 10 million particles. In one aspect, the first material according to the invention is apatite or hydroxyl apatite.

The particles according to the invention may be in range of micro-particles or nanoparticles. The size of these particles may be e.g. The particle size is preferably e.g. less than 200 μm, such as less than 100 μm, less than 50 μm, less than 35 μm, less than 20 μm or less than 10 μm. Moreover, the particles may be between about 0.1 and about 50 μm. In another aspect the microparticles may be in range of about 1 μm to about 500 μm, such as .e.g about 1 μm to about 100 μm, about 1 μm to about 50 μm, about 1 μm to about 25 μm, about 1 μm to about 15 μm, about 1 μm to about 10 μm, or about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 500 μm etc.

In one aspect, the particles of the first material is in range of e.g. about 0.5 μm to about 50 μm.

In one aspect, the particles of the first material is in range of e.g. about 1 μm to about 25 μm.

In one aspect, the particles of the first material is in range of e.g. about 1 μm to about 10 μm.

In a preferred embodiment, the drug may be administered once and once only and the particles in the first material are micro sized particles. As mentioned herein, the first material may be e.g. hydroxyapatite (HA) in any configuration.

As mentioned herein, the administered drug or drugs may be administered several times at different occasions as deemed necessary by the clinicians.

In yet a further aspect, the particle size may be in range of e.g. about 1 nm to about 200 nanometres (nm), such as e.g. less than about 150 nm, less than about 100 nm, less than about 50 nm, less than about 45 nm, less than about 40 nm, less than 35 nm, less than about 30 nm, less than about 25 nm, less than about 20 nm, less than about 15 nm, or less than about 10 nm. In one aspect the particle size is in range of e.g. about 1 nm to about 100 nm. In a further aspect, the particles of the first material may be about 1 nm, such as e.g. 5 nm, such as e.g. about 10 nm, such as e.g. about 15 nm, such as e.g. about 20 nm, such as e.g. about 25 nm, such as e.g. about 30 nm, such as e.g. about 35 nm, such as e.g. about 40 nm, such as e.g. about 45 nm, such as e.g. about 50 nm, such as e.g. about 100 nm, such as e.g. about 150 nm, or such as e.g. about 200 nm.

In one aspect, the particle size may be in range of about 40 nm to about 100 nm.

In a further aspect, the particle size may be in range of 10 nm to about 50 nm.

It has been surprisingly discovered by the inventors of present invention that in such instances that multiple administration of a drug or drugs (pharmaceutical compounds) is deemed necessary, the affinity towards nano-sized particles in the first material is higher for nano-sized particles in the sense that upon additional administration (i.e. repeated administration) of one or more pharmaceutical compositions, nano-sized particles of the first material displays a higher affinity than larger micro-sized particles. Thus, repeated administration of one or more pharmaceutical compounds may be employed as a means to re-load the nano-sized particles in the first material. This also has a benefit, in that nano-sized particles has the ability to enter a cell, such as e.g. a bacterial cell, and thus the local concentration of a pharmaceutical compound may be higher than in the systemic circulation and may thus attain concentrations equal to or above clinically relevant amounts to enable successful treatment of e.g. a bacterial infection.

In another aspect, the first material according to the invention may be a composition comprising a mixture or micro-sized particles and nano-sized particles. Thus the first material may comprise at least partly particles in the range of about 1 μm to about 500 μm, such as .e.g about 1 μm to about 100 μm, about 1 μm to about 50 μm, about 1 μm to about 25 μm, about 1 μm to about 15 μm, about 1 μm to about 10 μm, or about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 500 μm etc, and/or particles in the range of e.g. 1 and 200 nanometres (nm), such as e.g. less than 100 nm, less than 50 nm, less than 35 nm, less than 20 nm or less than 10 nm. In one aspect the particle size is in range of e.g. about 100 nm to about 1 nm.

Thus, in one aspect, the composition according to the invention may comprise only a first material as disclosed herein. As mentioned herein the first material may be e.g. hydroxyapatite (HA). The first material may be a mixture of micro-sized particles or nano-sized particles as mentioned herein, or alternatively may be a mixture of micro-sized particles and nano-sized particles, and thus a combination of micro-sized particles and nano-sized particles. For example, micro-sized particles may be in range of about 1 μm to about 500 μm, or about 1 μm to about 10 μm, and the nano-sized particles may be in range of about 1 nm to about 200 nm, or about 40 nm to about 100 nm.

In another aspect, present invention relates to a composition comprising first and a second material as disclosed herein. The first material may be a mixture of micro-sized particles and/or nano-sized particles as mentioned herein. In one example, the first material may be a mixture of micro-sized particles and nano-sized particles as mentioned herein, and consequently may be a mixture of micro-sized particles or nano-sized particles, or a combination of micro-sized particles and nano-sized particles. For example, micro-sized particles may be in range of about 1 μm to about 500 μm, or about 1 μm to about 10 μm, and wherein the nano-sized particles may be in range of about 1 nm to about 200 nm, or about 40 nm to about 100 nm, or about 10 nm to about 50 nm.

In a particular aspect, the composition may comprise a first material (which may be e.g. HA) and a second material (which may be e.g. CaS or calcium phosphate (CaP)), wherein the first material has a particle size of about 10 μm and/or 1 nm to about 50 nm, or about 50 nm. As mentioned herein, the first material may be embedded or dispersed in the second material.

Various ratios and relations between the amount of micro-sized and nano-sized particles may be present in a composition according to the invention. For example, the relation between the various particles sizes may be in range of about 1% (wt %) to about 99.9% (wt %) between nano:micro-sized particles, such as e.g. about 10% to about 90%, about 15% to about 85%, about 20% to about 80%, about 25% to about 75%, about 30% to about 70%, about 35% to about 65%, about 40% to about 60%, about 45% to about 55%, or about 50% to about 50%, about 55% to about 45%, about 60% to about 40%, about 65% to about 35%, about 70% to about 30%, about 75% to about 25%, about 80% to about 20%, about 85% to about 15%, about 90% to about 10%, about 95% to about 5%, or about 99.9% to about 1%. In one aspect, the ratio or relationship is about 50% to about 50% between nano:micro-sized particles. In one further aspect, the invention relates to a composition according to the invention wherein about 33.3% are micro-sized particles, 33.3% are nano-sized particles, and the remaining about 33.3% may be midsized particles which are not overlapping with the particle size intervals of the micro-sized and nano-sized particles.

In another aspect, the inventors of present invention have found that a certain amount of the first material is capable of binding a certain amount of the administered drug or drugs. In one non-limiting example, the first material may be hydroxyapatite. The amount of the first material may be e.g. at least about 1 mg, such as e.g. at least about 5 mg, such as e.g. at least about 10 mg, such as e.g. at least about 15 mg, such as e.g. at least about 20 mg, such as e.g. at least about 25 mg, such as e.g. at least about 30 mg, such as e.g. at least about 35 mg, such as e.g. at least about 40 mg, such as e.g. at least about 45 mg, such as e.g. at least about 50 mg, or at least about 500 mg. Alternatively, the amount of the first material may be in range of about 1 mg to about 50 mg, such as e.g. 5 mg to about 40 mg, such as e.g. about 5 mg to about 35 mg, such as e.g. about 5 mg to about 30 mg, such as e.g. about 5 mg to about 25 mg, such as e.g. about 5 mg to about 20 mg, such as e.g. about 5 mg to about 15 mg, such as e.g. about 5 mg to about 10 mg. As a further alternative, the amount of the first material in a composition according to present invention may be about 5 mg, such as e.g. about 10 mg, such as e.g. about 15 mg, such as e.g. about 20 mg, such as e.g. about 25 mg, such as e.g. about 30 mg, such as e.g. about 35 mg, such as e.g. about 40 mg, such as e.g. about 45 mg, such as e.g. about 50 mg.

It has been found that micro-sized as well as nano-sized particles of the first material, which in one aspect may be hydroxyapatite (HA), in the amount of about 5 mg is capable of binding about 10% of the administered drug. It has further been found that 10 mg of the first material is capable of binding about 30% of the administered drug and that 20 mg of the first material is capable of binding about 75% of the administered drug. In another aspect, it has been found that about 30 mg to about 40 mg is capable of binding nearly 100% of the administered drug. Thus, in one aspect the amount of the first material is preferably in range of about 30 mg to about 40 mg. The amount of the first material is to be understood as the amount being optionally mixed with the second or even third material, that may be in other ranges of amounts, in a composition according to the invention.

The ratio between the first and the second material may be in range of about about 1% to about 99.9% between first material:second material, such as e.g. about 10% to about 90%, about 15% to about 85%, about 20% to about 80%, about 25% to about 75%, about 30% to about 70%, about 35% to about 65%, about 40% to about 60%, about 45% to about 55%, or about 50% to about 50%, about 55% to about 45%, about 60% to about 40%, about 65% to about 35%, about 70% to about 30%, about 75% to about 25%, about 80% to about 20%, about 85% to about 15%, about 90% to about 10%, about 95% to about 5%, or about 99.9% to about 1%. In one aspect, the composition comprises only a first material. In one aspect, the ratio or relationship is about 40% to about 60% between first material:second material. In one non-limiting example, the first material may be e.g. HA (hydroxyapatite) in an amount of 40% (wt %) and the second material may be e.g. CaS (calcium sulphate) in an amount of 60% (wt %).

In one aspect, the first material such as e.g. hydroxyapatite is provided as hydroxyapatite particles (e.g. sintered hydroxyapatite particles) to be mixed with the second material such as e.g. calcium sulphate powder and water for the calcium sulphate to set, whereby the calcium phosphate particles becomes embedding in the calcium sulphate phase after setting. The water may be seen as a non-limiting example of a third material. A third material may be any additional solvent or further solid substance acting as diluent or mixing agent to the first and optionally the second material.

The composition according to the invention may comprise a first and optionally a second material. The first material may be administered as a particulate material which may optionally be in form of an emulsion, dispersion, solution etc. In such case, optionally a further diluent is added which acts as a carrier to the first material.

In another aspect, the composition according to the invention comprises a first and a second material. The composition may then be prepared by conventional mixing known in the art, which may further comprise addition of a solvent or other diluent. In one example, the mixing of the components of the composition may take place at normal room temperature and/or under normal atmospheric pressure. Alternatively, the mixing may take place under vacuum or reduced atmospheric pressure. It has been observed that the wettability affecting chemical binding of the finished composition is increased by mixing under vacuum or reduced atmospheric pressure.

In one aspect, the invention relates to a method of loading one or more pharmaceutical compounds onto the first and/or second material of present invention. Such method may comprise the steps of:
a) providing a particulate first material and/or a second material,
b) contacting/incubating/soaking one or more pharmaceutical compounds with the material in a),
c) separating non-bound pharmaceutical compounds from the material obtained in step b).

The one or more pharmaceutical compounds may be same or different and may have different affinities for the first and second material. The one or more pharmaceutical compounds may be dissolved in any suitable solvent such as a physiological saline solution, water or other aqueous solution, buffered solution, or any suitable organic solvent or any mixtures thereof.

In a non-limiting example, the composition according to the invention may be e.g. in form of a pellet. The pellet may be prepared by mixing the first material with the second material (such as e.g. mixing Ha with CaS) in a powder form, in a suitable solvent such as e.g. a saline aqueous solution (0.9 wt % NaCl in water; known as normal saline), and/or hyaluronic acid, and/or iodine-based mixing solution. The resulting slurry may be supplied with one or more pharmaceutical compounds, such as e.g. one or more ansamycins, and/or one or more glycopeptides, and/or one or more aminoglycosides.

In one aspect, the resulting slurry above is supplied with one or more ansamycins.

In a further aspect, the resulting slurry above is supplied with one or more glycopeptides.

In yet a further aspect, the resulting slurry above is supplied with one or more aminoglycosides.

In one aspect, the resulting slurry above is supplied with vancomycin. The slurry may be employed as a liquid or semi-solid composition and may be injected into the subject. Alternatively, the slurry may be allowed to cure into a solid composition and may be implanted into the subject.

In one aspect, the resulting slurry above is supplied with gentamicin. The slurry may be employed as a liquid or semi-solid composition and may be injected into the subject. Alternatively, the slurry may be allowed to cure into a solid composition and may be implanted into the subject.

In one aspect, the resulting slurry above is supplied with rifampicin. The slurry may be employed as a liquid or semi-solid composition and may be injected into the subject. Alternatively, the slurry may be allowed to cure into a solid composition and may be implanted into the subject.

As implied herein, the above mentioned, the composition for use according to the invention, may thus comprise one or more pharmaceutical compounds, wherein the use comprises further administering one or more pharmaceutical compounds. For example, the composition may comprise e.g. vancomycin or gentamicin, wherein the composition is administered to a subject in need.

Thereafter, an additional pharmaceutical compound may be administered to the subject. The additional pharmaceutical compound may be administered locally near the implanted or administered composition or systemically to the subject. A non-limiting example of an additional pharmaceutical compound may be e.g. rifampicin.

The period during which the one or more pharmaceutical compounds is contacted with the particulate first material and/or a second material, may be within range of minutes, hours or days, such as e.g. about 1 hour, such as e.g. about 3 hours, such as e.g. about 6 hours, such as e.g. about 12 hours, such as e.g. about 24 hours, such as e.g. about 48 hours etc.

After contacting/incubating the one or more pharmaceutical compounds with the particulate first material and/or a second material, the resulting mixture may be separated from the solution or suspension comprising the one or more pharmaceutical compounds by centrifugation or filtration etc. the resulting particulate material may be rinsed with any suitable solvent and optionally thereafter allowed to dry.

As is apparent form present invention, the composition according to the invention may be regarded as an extended release composition, such that the one or more, or two or more pharmaceutical compounds are released from the first which may optionally comprise a second material, are continuously released from the said first and second material (if present) in a prolonged manner or over an extended period of time. Such an extended period of time may be e.g. at least 12 h, or such as e.g. at least 1 day, e.g. at least 2 days, e.g. at least 3 days, e.g. at least 5 days, e.g. at least 7 days, e.g. at least 14 days, e.g. at least 21 days, e.g. at least 28 days, e.g. at least 35 days, e.g. at least 2 months, e.g. at least 3 months etc. after administration of the composition according to the invention.

Thus according to the invention, it may be sufficient to provide the subject, i.e. administer the composition once during a period of 12 h, or 1 day, 2 days, 3 days, 5 days, 7 days, 14 days, 21 days, 28 days, 35 days, or 2 months, e.g. or 3 months etc.

In a different aspect, the pharmaceutical compounds may be administered separately several times during the treatment period. Administration may be locally (near the implanted HA/CaS matrix) and/or systemically via any suitable route.

Consequently, present invention provides a composition allowing for a continuous release of the antibiotic compounds present in the composition.

In one aspect, the composition may comprise about 50 mg to about 5000 mg of HA/CaS.

In a non-limiting example, the composition may comprise about 100 mg to about 2000 mg, or such as e.g. about 300 mg to about 1000 mg, or about 500 mg of HA/CaS.

In a further aspect, the composition may comprise one or more pharmaceutical compounds in a clinically relevant amount. In a particular aspect, such clinically relevant amount may be in any range of from about 0.5 mg to about 2000 mg. In a non-limiting aspect, the range/amount of e.g vancomycin may be in range of about 1 mg to about 50 mg, such as e.g. about 25 mg.

In a further non-limiting aspect, e.g. gentamicin may be present in an amount of about 1 mg to about 50 mg, such as e.g. about 25 mg.

In yet a further non-limiting aspect, e.g. rifampicin may be present in an amount of about 0.1 mg to about 20 mg, such as e.g. about 8 mg.

Importantly, the inventors of present invention have surprisingly found that even after a prolonged treatment employing the composition according to present invention, no bacterial resistance arises. One non-limiting examples is seen in e.g. rifampicin resistant bacteria, where no build-up of antibiotic resistance is seen even after 35 days after administration of the composition according to the invention.

As mentioned herein, the composition according to the invention may comprise one or more pharmaceutical compounds, such as one or more antibacterial compounds or antibiotic compounds, such as e.g. vancomycin or gentamicin. In one aspect, the composition may be administered to a subject in need thereof. Thereafter a further pharmaceutical compound may be administered to the subject such as e.g. rifampicin. The further pharmaceutical compound may be administered to the subject in any suitable manner or mode of administration such as e.g. by injection locally near the site in need of being treated, or may be administered systemically into e.g. the circulatory system of the subject. The further pharmaceutical compound may be administered simultaneously with the composition according to the invention or may be administered about 6 h after administration of with the composition according to the invention, such as e.g. about 12 after administration of the composition, such as e.g. about 24 after administration of the composition, such as e.g. about 2 days after administration of the composition, such as e.g. about 3 days after administration of the composition, such as e.g. about 4 days after administration of the composition, such as e.g. about 5 days after administration of the composition, such as e.g. about 6 days after administration of the composition, such as e.g. about 7 days after administration of the composition, such as e.g. about 14 days after administration of the composition, such as e.g. about 21 days after administration of the composition, such as e.g. about 28 days after administration of the composition, such as e.g. about 35 days after administration of the composition.

The further pharmaceutical compound may be administered once during the above mentioned period. Alternatively, the further pharmaceutical compound may be administered one or more times after the administration of the first administration, wherein the further administration takes place once daily after the first administration, such as once daily for e.g. about 1 day, about 2 days, about 3 days, about 4 days, about 5 days, etc.

The invention is further illustrated in the following non-limiting examples.

### Examples

### Materials and Methods

### Materials Used

Medical grade rifampicin (RIF) (Rifampicin Ebb, 600 mg; Sanofi S.p.A, Anagni, Italy) was purchased from the local pharmacy (Apoteket AB, Sweden). CaS/HA composite containing GEN (gentamicin) (Cerament® G) or VAN (Vancomycin) (Cerament® V) were supplied by Bonesupport AB, Lund, Sweden. The particle size of the hydroxyapatite particles in the Cerament compositions are in range of about 1 μm to about 10 μm. Phosphate buffered saline (PBS, pH 7.0-7.2) was supplied by Cytiva, Utah, US. Sodium pyruvate were purchased from Thermo scientific, U.S.A. Heat inactivated fetal bovine serum (FBS), trypsin-EDTA solution (0.25%), sodium pyruvate solution, and 3-(4,5-Dimethylthiazol-2-yl)-2,5-diphenyltetrazolium bromide (MTT) reagent were purchased from Sigma Aldrich, Germany. MC3T3-E1 subclone 4 cells were purchased from ATCC (VI, U.S.A). α-minimum essential medium (αMEM) without phenol red was purchased from Life Technologies Europe BV (Stockholm, Sweden).

### Preparation and Characterization of Antibiotic Loaded CaS/HA Composites

Antibiotic loaded CaS/HA composite synthesis: The following combinations of CaS/HA and antibiotics were prepared: CaS/HA-VAN, CaS/HA-VAN+RIF, CaS/HA-GEN, CaS/HA-GEN+RIF, and CaS/HA-RIF. For CaS/HA-VAN, CaS/HA-VAN+RIF composites, 500 mg of CaS/HA powder (Cerament V, Bonesupport AB, Sweden) was hand-mixed in a 48-well plate with either only VAN (24.57 mg) or in combination of RIF (8.11 mg) dissolved in 215 μL mixing-solution (C-TRU, Bonesupport AB, Sweden) using a sterile wooden stick for 30 seconds at 22±1° C. both in clockwise and anticlockwise directions after every 3 rounds. The paste was transferred to a 1 mL syringe (B BRAUN, Melsungen, Germany) connected to an 18 G needle (0=1.2 mm, L=4 cm) and extruded into a mould with hemispherical wells (0=4.8 mm) to obtain uniform sized pellets for evaluating antibiotic release profile, material degradation and continuous Kirby-Bauer disk diffusion assay. CaS/HA-GEN or CaS/HA-RIF composites were prepared by mixing 500 mg of CaS/HA powder (Cerament G, Bonesupport AB, Sweden) and GEN (24.57 mg) or RIF (8.11 mg) dissolved in 283.5 μL mixing-solution (Bonesupport AB, Sweden). For CaS/HA-GEN+RIF composites, preparations were the same but with the difference of intermittent mixing technique. Briefly, 500 mg of CaS/HA powder (Cerament G, Bonesupport AB, Sweden) was first hand-mixed with GEN (24.57 mg) dissolved in 200 μL mixing-solution (Bonesupport AB, Sweden) and 30 seconds later, RIF (8.11 mg) dissolved in 83.5 μL mixing-solution (Bonesupport AB, Sweden) added and mixed again. This intermittent mixing technique was adopted to prevent the rapid setting of the composite when GEN and RIF mixed simultaneously. CaS/HA-GEN, CaS/HA-RIF and CaS/HA-GEN+RIF hemispherical pellets were prepared as mentioned previously.

### Injectability and Setting Time

CaS/HA composites were prepared as mentioned above. The injectability of the composite was then evaluated by extruding 250 μL of the paste manually through a graduated 1 mL syringe connected to an 18 G needle (0=1.2 mm, L=4 cm) into a mould with hemispherical wells (0=4.8 mm). Total weight of the syringe with paste before and after extrusion were measured. To normalize the amount of composite that remained in the needle and syringe tip after injection, the mean of the paste extruded from the syringe at the first time point (for GEN, 4 min; VAN and RIF, 3 min) was calculated for the tested concentrations (normalized value). Injectability (%) was calculated by dividing the weight of the paste extruded from the syringe with a normalized value. Each test was repeated for three times at various time points (GEN: 4, 6 and 8 min; VAN and RIF: 3, 5, and 7 min). The composite extruded into the mould wells was left to solidify as per manufactures recommendations for 20 min and to confirm the setting of the tested composites, the consistency of the pellets were evaluated manually.

$$Injectability ( \% ) = \frac{weight of the paste extruded from the syringe}{normalized value} \times 100$$

### Antibiotic Release Profile

For each combination, pellets in triplets were placed in 2 mL microcentrifuge tube containing 1 mL of sterile PBS and placed at 37° C. At different time points (Day 1, 3, 7, 14, 21, 28 and 35) PBS was collected and tubes were supplemented with fresh 1 mL PBS. The amount of VAN and RIF released from the pellets were quantified using liquid chromatography-tandem mass spectrometry (LC-MS/MS) method (Thermo Fisher Scientific, USA) and GEN quantification was done by cloned enzyme donor immunoassay (CEDIA) on Roche Cobas, C501 (Roche Diagnostics, Risch-Rotkreuz, Switzerland).

### Testing of Antibacterial Effects

### Bacterial Strains and Culture Conditions

For in-vitro bacterial experiments, Staphylococcus aureus standard strain obtained from American Type Culture Collection (Staphylococcus aureus ATCC® 25923™), and S. aureus clinical strain P3, isolated from a case of prosthetic joint infection at Lund University Hospital were used. The strains were preserved in −80° C. and before experiments subcultured onto in-house made sheep blood agar plates. Prior to use, a minimum of two subcultures were done.

### Efficacy on Planktonic Bacteria by Continuous Kirby-Bauer Disk Diffusion Assay

The antimicrobial efficacy of composites on planktonic bacteria were tested by Kirby-Bauer disk diffusion assay. For tested combinations of CaS/HA-antibiotics, pellets in triplets were used for each strain of S. aureus. The bacteria were inoculated into sheep blood agar plates and incubated at 37° C. for 24 h. Then, bacterial suspensions were prepared by dissolving the bacteria in sterile saline (NaCl 0.9%) to an optical density (O.D.) 600=0.1±0.005 as measured by spectrophotometer (Thermo Scientific GENESYS 20). By lawn culture, bacteria were inoculated on in-house made Mueller-Hinton agar (MHA) plates and using sterile forceps, pellets were transferred to the inoculated plates within 3-15 min of inoculation. The plates were then incubated at 37° C. for 24 h, and diameter of the zone of inhibition (ZOI) was measured using a standard ruler. After measuring the ZOI, the pellets were transferred to new set of plates inoculated with bacteria using sterile forceps, and the procedure repeated until their zone of inhibition became zero, or for a maximum of 28 days. At predetermined time points (day 1, 3, 7, 14, 21, and 28) MHA plates exhibiting ZOI were imaged using ChemiDoc™ MP imaging system (Bio-Rad Laboratories, Hercules, CA). For GEN and RIF pellets, using CLSI guidelines, ZOI of 15 and 20 mm respectively taken as strong antibacterial effect, and an ZOI of less than these were represented moderate antibacterial effect. For VAN, ZOI of 17 mm used as surrogate for strong antibacterial effect.

Antibacterial effect of pellets after day-35: To mimic the in vivo conditions, after day-35, the antibacterial of the pellets that have been used for antibiotic release assay were tested in a similar way as mentioned above. Briefly, following the day-35 fraction collection for antibiotic release assay, the pellets were crushed to form a paste, and using a sterile spatula, it was transferred to the MHA plates inoculated with S. aureus ATCC 25923. After 18 h of incubation, the ZOI (Zone Of Inhibition) were noted and plates were imaged using ChemiDoc™ MP imaging system (Bio-Rad Laboratories, Hercules, CA).

Efficacy on preformed biofilms: Using a modified biofilm quantification method, the antibiotics released at different time points (Day 1, 3, 7, 14, 21, 28 and 35) from the pellets of CaS/HA-antibiotic composites were tested for their ability to disrupt preformed biofilm. S. aureus were grown in tryptic soy broth supplemented with 1.0% glucose (TSBG) in a rotary shaker (180 rpm) at 37° C. for 16-18 h. Then, the Bacterial suspension were centrifuged at 4000 rpm for 10 min and immediately resuspended in fresh TSBG. The resuspended bacterial suspension were diluted to optical density 0.17 measured at 600 nm (Thermo Scientific GENESYS 20). From the diluted suspension, 11.4 μL were added to 138.6 μL of TSBG in the wells of 96-well flat bottom tissue culture treated polystyrene microtitre plate (Costar, Kennebunk, ME, USA) coated with poly-L-lysine (0.2 mg/mL) (Sigma-Aldrich, Germany) and incubated at 37° C. statically. Biofilm were allowed to form on the wells for 48 h with change of media after 24 h of incubation. After incubation, biofilms were gently washed three times with sterile PBS to remove the planktonic bacteria. Thereafter, 200 μL of TSBG and TSBG-antibiotic fractions mixture (1:1) were added to the control and test wells respectively, and incubated at 37° C. for 24 h. The wells were then gently washed two times with PBS and fixed with 150 μL of methanol for 20 min. After air drying for overnight, the remaining biofilms were stained with 150 μL 1% (w/v) crystal violet solution (Sigma-Aldrich, Germany) for 15 min at room temperature. The wells were washed three times with PBS and air dried before adding 95% ethanol to resolubilize the dye bound to cells. After 30 min, 100 μL from each well was transferred to new 96-well plate and optical density was measured at 590 nm using a microplate reader (iMark™, Bio-Rad, Japan). The abosrbance from the test wells which correlates to the amount of biofilm remained after exposure to the antibiotic fractions, were compared with the absorbance of untreated control wells. The efficacy of the antibiotic fractions were tested on both S. aureus ATCC 25923, and S. aureus clinical strain P-3. For each tested group of antibiotic fractions, five separate wells were used (n=5).

### Scanning Electron Microscopy of Biofilms

As mentioned in above, biofilms were first allowed to form on glass coverslips (Ø=13 mm) coated with poly-L-lysine (0.2 mg/mL) for 48 h in a 12-well plate (Costar, Kennebunk, ME, USA) with change of media after 24 h of incubation.

After incubation, biofilms were gently washed three times with sterile PBS to remove the planktonic bacteria. Thereafter, 700 μL of TSBG and TSBG-antibiotic fractions mixture (1:1) were added to the control and test wells respectively, and incubated at 37° C. for 24 h. The wells were then gently washed two times with PBS and fixed in 2.5% glutaraldehyde in 0.15 M sodium cacodylate buffer (pH 7.4) for overnight at room temperature. Fixed samples were washed with 0.15 M sodium cacodylate (pH 7.4) for 10 min to a minimum of four cycles, and dehydrated with increasing concentrations of ethanol followed by critical point drying with liquid carbon dioxide in Baltec CPD030, using absolute ethanol as the intermediate solvent. Samples were then mounted onto aluminium holders and sputter coated with 20 nm gold/palladium in Leica AC200, and examined under a DELPHI Phenom-World microscope (EM unit, Infection Medicine, Lund University).

Viable Bacteria after Continuous Treatment with Antibiotic Fractions, and Development of Resistance to VAN (Vancomycin), GEN (Gentamicin) and RIF (Rifampicin)

Viable bacteria after continuous treatment with antibiotic fractions, and the development of bacterial resistance to VAN, GEN and RIF were determined by exposing the biofilm embedded bacteria continuously to release fractions of antibiotics from CaS/HA-VAN/GEN pellets with or without RIF from predetermined time points (Day 1, 3, 7, 14, 21, 28 and 35). Briefly, 48 h-old S. aureus biofilms were established on 96-well micro-titre plates as mentioned above. Biofilms were then gently washed two times with sterile PBS. The biofilms in the control and test wells were exposed to 200 μL of plain TSBG and TSBG-antibiotic fractions mixture (1:1) respectively for 16 h, followed by 8 h incubation in 150 μL of antibiotic-free TSBG. This cycle was repeated until antibiotic fractions from all time points were exposed: a total of seven days. Thereafter, each well were gently washed two times with sterile PBS. To remove the remaining biofilms from the wells, 200 μL sterile PBS was added, and wells were scraped with sterile plastic pipette tips followed by sonication of the micro-titre plate for 5 min in an ultrasonic bath (Elmasonic S 30H; Elma Hans Schmidbauer GmbH & Co. KG, Singen, Germany). After serial dilution, the number of CFUs were determined by plating on both antibiotic-free Tryptic Soy Agar (TSA), and TSA supplemented with varying concentrations of antibiotics (VAN/GEN: 4-24 μg/plate; RIF: 1-32 μg/plate). The development of resistance was tested on both S. aureus ATCC 25923, and S. aureus clinical strain P-3 whose MIC values against VAN, GEN and RIF were predetermined using MALDI-TOF MS system (Vitek MS™; BioMe'rieux, France) (S. aureus ATCC 25923: VAN=2 μg/mL) GEN=0.064 μg/mL, RIF=0.008 μg/mL; S. aureus clinical strain P-3: VAN=1 μg/mL, GEN=0.5 μg/mL, RIF=0.008 μg/mL). Strains showing resistance to RIF were further confirmed using MALDI-TOF MS system. For each tested group of antibiotic fractions, four separate wells were used (n=4).

### Statistical Analysis

All data was represented as mean±SD or SEM. A student t-test was used to analyze injectability of CaS/HA-VAN/GEN with or without RIF, material degradation of the pellets, MTT assay, and to compare the differences in biofilm destruction. A student t-test or one-way ANOVA with Dunn's multiple comparisons test was used to analyze the cumulative release profile of VAN, GEN or RIF. For comparison of difference in ZOI between different groups, ANOVA with Tukey's multiple comparison was used. A p<0.05 was considered statistically significant. Details of statistical analysis are indicated in each figure legend. All data processing was carried out on GraphPad Prism version 9.1.2 for MacBook (GraphPad Software, San Diego, CA, USA).

### Results

### Preparation and Characterization of Antibiotic Loaded CaS/HA Composites

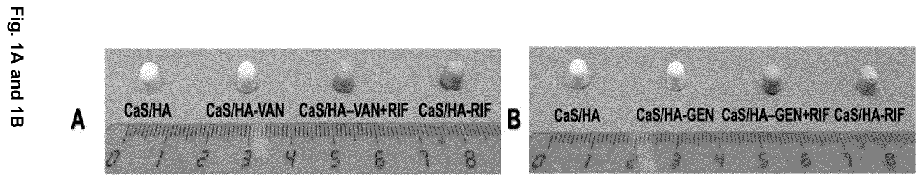

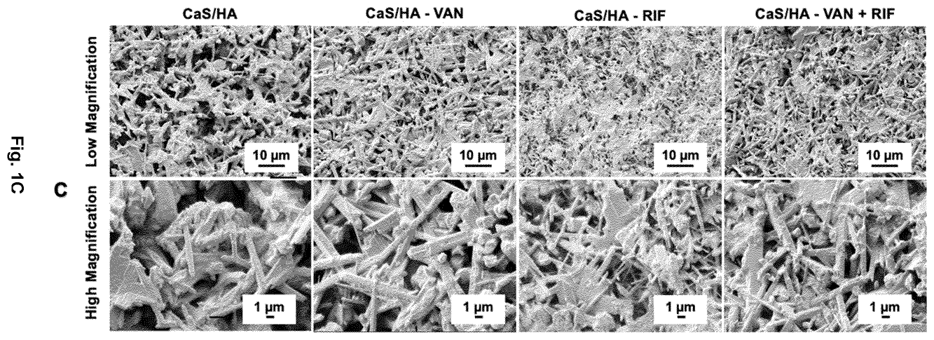

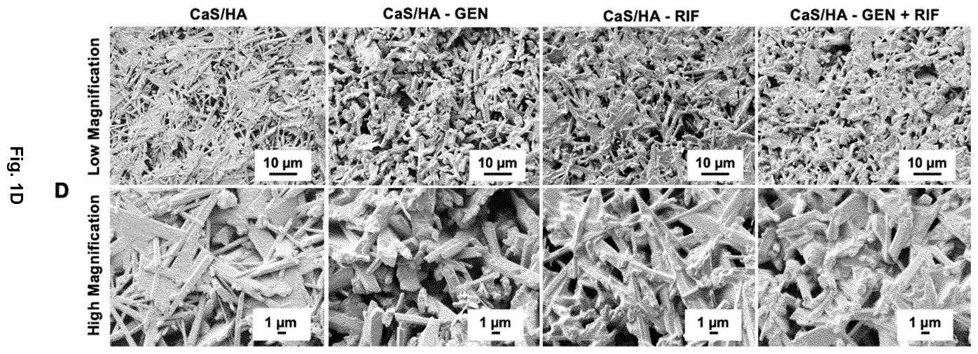

Pellets of all tested combinations of antibiotic loaded CaS/HA composites were characterized for the morphology and physio-chemical properties. CaS/HA pellets with either GEN or VAN were white in colour while combinations of RIF had dark orange colour (FIGS. 1A & B). SEM revealed that the addition of RIF to CaS/HA-VAN/GEN composites appears to have no effect either in the surface morphology or pore structure (FIGS. 1C & D). FTIR characterization further confirmed that the RIF was loaded onto CaS/HA-VAN/GEN composites. CaS/HA-VAN+RIF showed the same transmittance peaks at 1584 cm⁻¹, 1559 cm⁻¹, 1430 cm⁻¹, 1225.3 cm⁻¹ and 889 cm⁻¹ as RIF, whereas CaS/HA-GEN+RIF showed the same transmittance peaks as that of RIF at 1558 cm⁻¹, 1433.4 cm⁻¹, 1372.2 cm⁻¹, 1225.3 cm⁻¹ and 891 cm-1, which could not be seen in only CaS/HA-VAN/GEN composites (FIGS. 2A & B). CaS/HA-VAN composites could be completely extruded from the syringes at all three tested time points (3, 5 and 7 min) whereas addition of RIF to CaS/HA-VAN significantly reduced the injectability, and by 5 min the composite became uninjectable (FIG. 2C). The CaS/HA-GEN composites could be completely extruded from the syringes at 4 min without blocking the syringe outlet or causing obvious pressure filtration effects, and by 8 min it dropped to almost 47% (FIG. 2C). By dropping the injectability to almost 12% by 6 min, a significant injectability reduction trend was observed with the addition of RIF to CaS/HA-GEN as well. At both 3 and 5 min, CaS/HA-RIF composites maintained the injectability and by 7 min it reduced to almost 11%. All the CaS/HA-antibiotics composites were completely set within 15 minutes of injection and they subjectively seemed durable and did not crumble while handled. All materials had a similar degradation rate, irrespective of the type of antibiotic added. Nearly 50% of the material degraded over a 1-month period (FIG. 2D).

### Antibiotic Release Profile

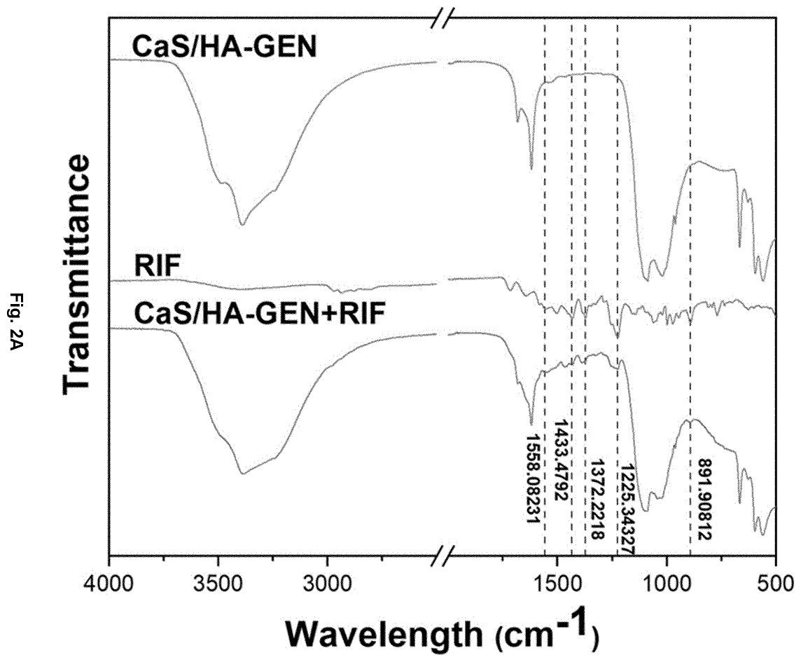

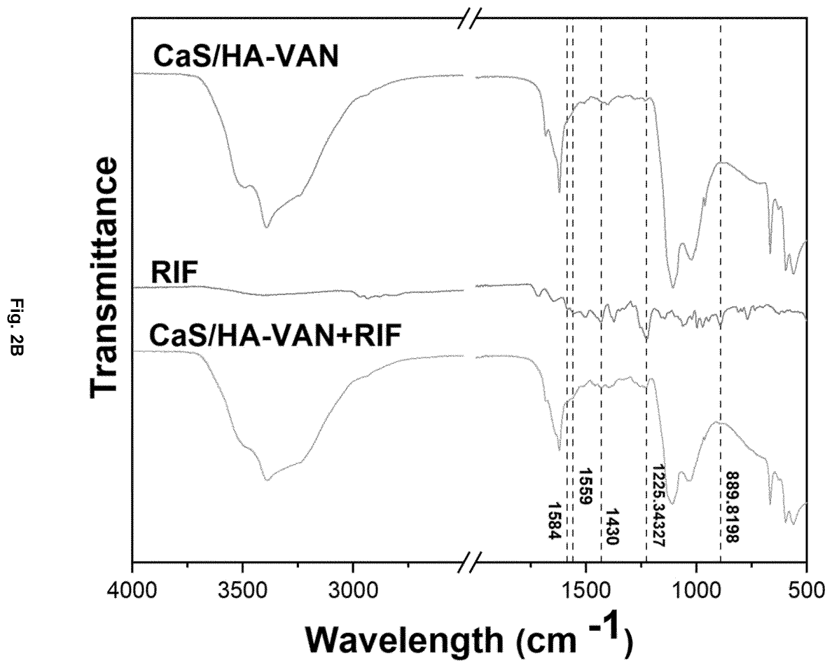

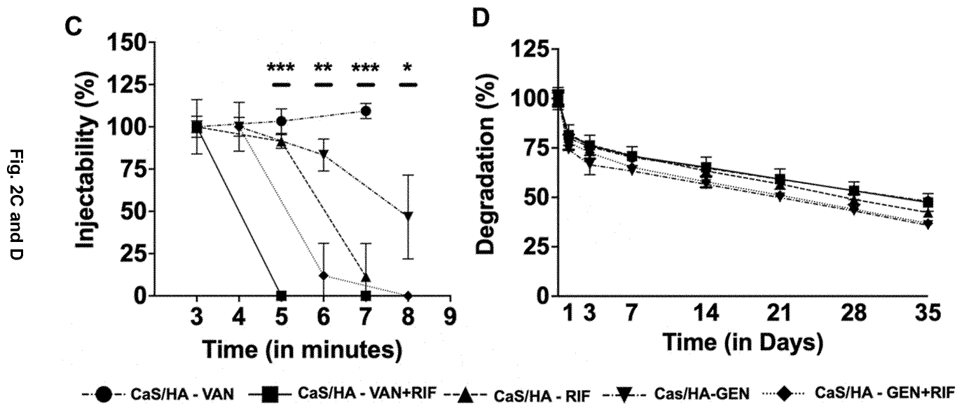

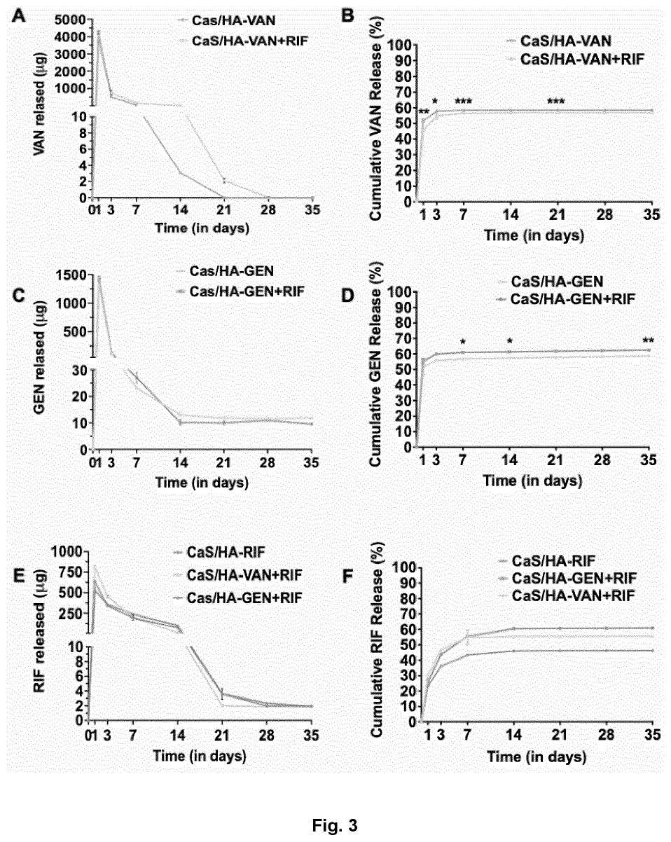

In the tested CaS/HA pellets of both VAN or combination of VAN+RIF, at D-1, 3, 7 and 21, there was a significant difference in the amount of VAN released per day but overall cumulative release profile of VAN remained almost unchanged (58% and 56.7% respectively) (FIGS. 3 A & B). Similar cumulative release trend was observed with GEN as well (CaS/HA-GEN, 58.6% and CaS/HA-GEN+RIF, 62.6%) (FIGS. 3 C & D). From CaS/HA-VAN and CaS/HA-VAN+RIF, VAN concentrations above the MIC levels of S. aureus were obtained till day-14 (3.08 μg/mL) and 21 (2.1 μg/mL) respectively. Till study end point at day-35, CaS/HA-GEN and CaS/HA-GEN+RIF pellets eluted GEN of concentrations well above the MIC levels of S. aureus; 11.8 μg/mL and 9.5 μg/mL respectively. In case of RIF, an increased cumulative release profile was obtained when it combined with CaS/HA-VAN (55.5%) or GEN (60.8%) compared to CaS/HA-RIF alone (46.3%) (FIGS. 3 E & F). On day-35, concentrations of RIF from CaS/HA-RIF (1.94 μg/mL), CaS/HA-VAN+RIF (1.87 μg/mL), and CaS/HA-GEN+RIF (1.87 μg/mL) all found to be above the MIC levels of S. aureus.

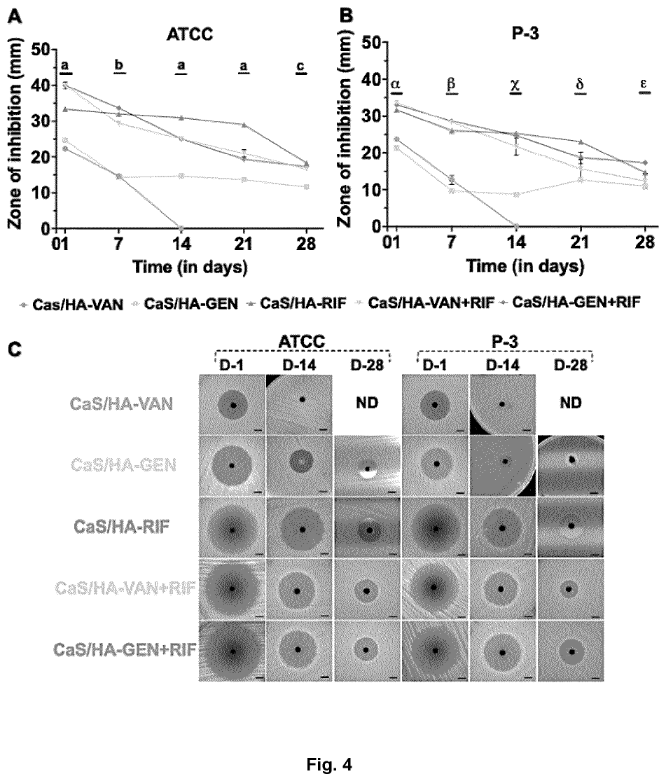

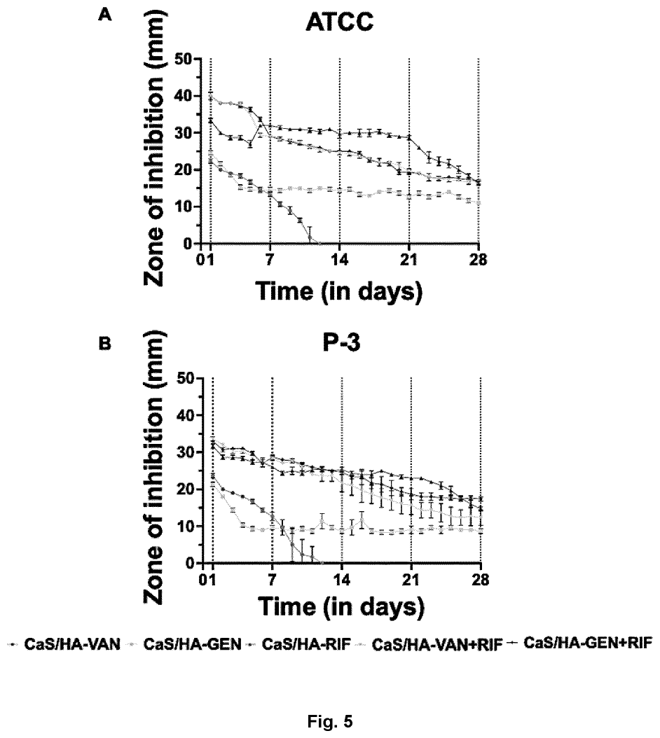

Efficacy on Planktonic Bacteria by Continuous Kirby-Bauer Disk Diffusion Assay There was a general trend that ZOI of all different composite pellets decreased over time (FIG. 4A-C, FIG. 5). Out of all the tested CaS/HA pellets, antibacterial effect of CaS/HA-VAN against both S. aureus strains declined sharply and showed no effect by day-12. CaS/HA-GEN pellets exhibited a strong antibacterial effect until day-15 and day-3 against S. aureus ATCC 25923 and clinical strain P-3 respectively. There after it maintained a low but almost steady antibacterial effect till day-28. Against S. aureus ATCC 25923, both CaS/HA-VAN/GEN+RIF combinations showed strong antibacterial effect till day-20 but in case of clinical strain P-3, it was day-16 for CaS/HA-VAN+RIF, and day-20 for CaS/HA-GEN+RIF. Compared to pellets of all combinations, the antibacterial effect persisted for a longer time for CaS/HA-RIF pellets against both S. aureus ATCC 25923 (until day-26) and clinical strain P-3 (until day-25).
Antibacterial Effect of Pellets after Day-35

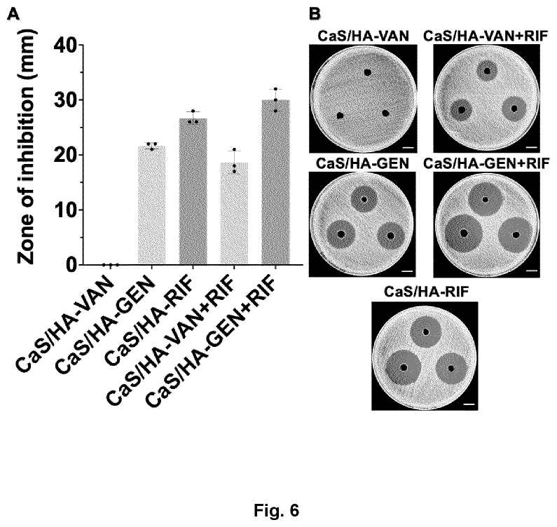

Except CaS/HA-VAN, all other tested CaS/HA-antibiotics groups shown strong antibacterial effect at day-35 (FIG. 6). By exhibiting larger ZOI of all groups, CaS/HA-GEN+RIF appears to have the strongest antibacterial effects.

### Efficacy on Preformed Biofilms

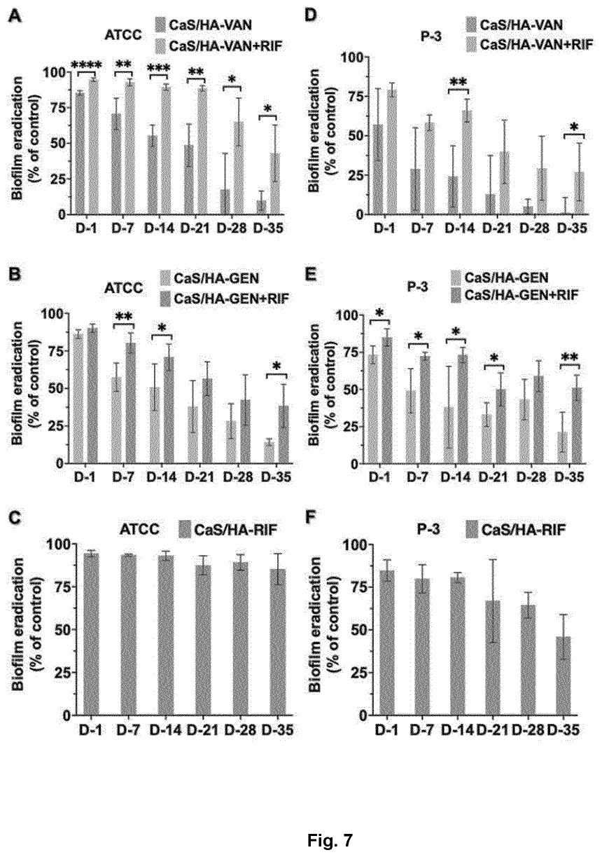

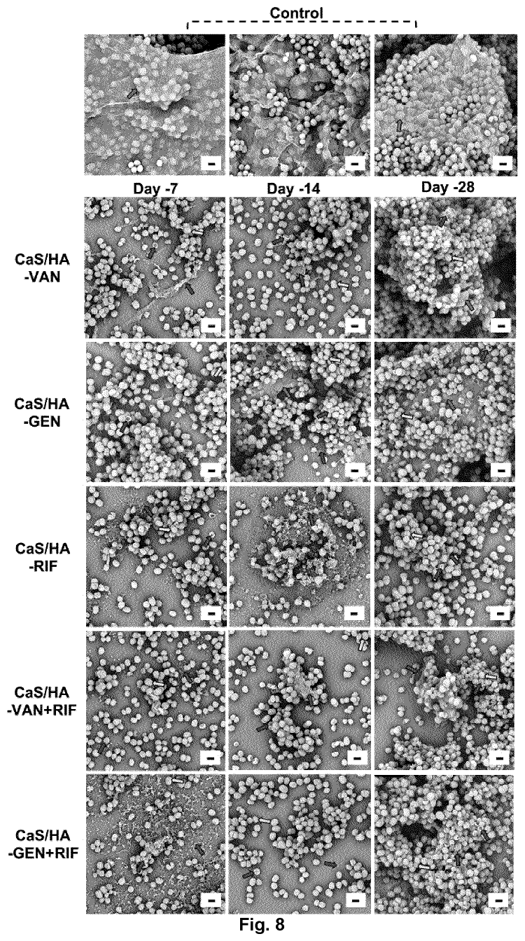

Overall, there was an excellent biofilm destruction trend was observed with CaS/HA-VAN/GEN+RIF combinations than CaS/HA-VAN/GEN alone (FIG. 7A-F). In comparison to CaS/HA-VAN, all tested fractions from CaS/HA-VAN+RIF shown significant destruction of S. aureus ATCC biofilms (FIG. 7A, D) but against clinical strain P-3 biofilms (FIG. 7B, E), only D-14 and D-35 achieved significant difference. Almost all tested fractions of CaS/HA-GEN with RIF showed significant biofilm destruction against clinical strain P-3 biofilms. Compared to CaS/HA-VAN/GEN alone, CaS/HA-RIF fractions exhibited strong antibiofilm property against both S. aureus ATCC and P-3 biofilms (FIGS. 7 C & F). SEM analysis of the preformed biofilm destruction also showed a comparable trend with that of CV staining results (FIG. 8).

### Testing for Development of Bacterial Resistance to VAN, GEN and RIF

Following continuous treatment of S. aureus ATCC 25923 and clinical strain P-3 biofilms with CaS/HA-VAN/GEN/RIF alone antibiotic fractions, the number of viable CFU's obtained on antibiotic-free TSA ranged from 0-6.5×10⁷, and 1.4-3.1×10⁸ CFU respectively (Table 1). But with complete destruction of biofilm embedded bacteria, no viable CFU's were obtained for CaS/HA-VAN/GEN+RIF combinations, and were clearly more potent in biofilm eradication than CaS/HA-VAN/GEN/RIF alone groups.

S. aureus ATCC and clinical strain P-3 biofilms that had been subjected to CaS/HA-RIF alone developed resistance to RIF, and were grew on plates with 32 mg/L of RIF indicating high level of resistance. This was further confirmed using MALDI-TOF. But, none of the tested biofilms exposed to RIF in combination with VAN/GEN developed resistance to RIF. Also, no resistance to VAN or GEN were noted for any of the CaS/HA-antibiotic combinations. Bacteria in control wells were sensitive to all tested antibiotics.

TABLE 1
Viable CFU's after continuous treatment with antibiotic
fractions, and development of rifampicin-resistant CFU's

| Antibiotic fractions | Bacteria tested, and | | | Rifampicin |
|---|---|---|---|---|
| from CaS/HA- | total CFU before | Remaining viable CFU's, | | resistant CFU's, |
| antibiotics pellets | treatment | median (range) | | median (range) |
| CaS/HA-VAN | S. aureus | 5.9 × 10⁷ | (5.6 × 10⁷-6.5 × 10⁷) | 0 |
| CaS/HA-GEN | ATCC25923, 6.5 × 10⁷ | 2.4 × 10⁷ | (1.4 × 10⁷-3.7 × 10⁷) | 0 |
| CaS/HA-RIF | | 1.1 × 10⁷ | (0-4.5 × 10⁷) | 1.8 × 10⁵ (0-7.5 × 10⁵) |
| CaS/HA-VAN + RIF | | 0 | | 0 |
| CaS/HA-GEN + RIF | | 0 | | 0 |
| CaS/HA-VAN | S. aureus clinical | 1.73 × 10⁸ | (1.4 × 10⁸-1.92 × 10⁸) | 0 |
| CaS/HA-GEN | strain P-3, 2.8 × 10⁸ | 2.54 × 10⁸ | (2.2 × 10⁸-3.1 × 10⁸) | 0 |
| CaS/HA-RIF | | 1.05 × 10⁸ | (2.5 × 10²-2.8 × 10⁸) | 9.2 × 10⁷ (0-1.9 × 10⁸) |

| | | |
|---|---|---|
| CaS/HA-VAN + RIF | 0 | 0 |
| CaS/HA-GEN + RIF | 0 | 0 |

1. A composition for use in the treatment or prevention of a bacterial infection in a subject, the composition comprising;
i) a first material being hydroxyapatite,
ii) a second material being calcium sulphate,
iii) two or more pharmaceutical compounds, wherein the at least two pharmaceutical compounds are selected from a combination of vancomycin and rifampicin, or a combination of gentamicin and rifampicin,
to thereby prevent or reduce or eliminate the occurrence of bacterial resistance against said the pharmaceutical compounds.

2. (canceled)

3. (canceled)

4. The composition for use according to claim 1, wherein the first material is hydroxyapatite in any configuration, and wherein the second material is calcium sulphate in any configuration, or crystal form, or type of hydration.

5. (canceled)

6. (canceled)

7. (canceled)

8. (canceled)

9. (canceled)

10. The composition for use according to claim 1, wherein the particle size of the first material is in range of from about 1 nm to about 500 μm.

11. The composition for use according to claim 1, wherein the bacterial infection is in relation to bone tissue, such as e.g. deep bone infection (DBI).

12. The composition for use according to claim 1, wherein the two or more pharmaceutical compounds are pre-mixed or pre-soaked into the first and second material prior to administration to the subject.

13. The composition for use according to claim 1, wherein the composition is administered to the subject by injection.

14. The composition for use according to claim 1, wherein the composition is administered as a solid formulation or as a fluid or semi-fluid formulation.

15. The composition for use according to claim 1, wherein the composition is administered by injection near or into bone tissue.

16. (canceled)

17. The composition for use according to claim 1, wherein no further pharmaceutical compounds capable of treating a bacterial infection are administered after administration of the composition according to claim 1.

18. The composition for use according to claim 1, wherein the one or more pharmaceutical compounds are released from the first and second material for an extended period of time such as e.g. at least about 1 day, at least about 3 days, at least about 7 days, at least about 14 days, at least about 21 days, at least about 28 days, or at least about 35 days or more.

19. (canceled)

20. A method of treating a subject, wherein the subject has a bacterial infection, and wherein the subject is administered a composition according to any one of the preceding claims to thereby prevent, or eliminate, or reduce, the occurrence of bacterial resistance.

21. The method according to claim 20, wherein the bacterial infection is caused by or related to rifampicin resistant bacteria.

22. The method according to claim 20, wherein the composition is administered once during a treatment period of about 1 day, or about 3 days, about 7 days, about 14 days, about 21 days, about 28 days, or about 35 days or more.

23. The method according to claim 20, wherein the composition is administered as a solid, semi-solid or as an injectable paste.

24. (canceled)

25. A method of preparing a composition for use according to claim 1, the method comprising the steps of
a) providing a particulate first material being hydroxyapatite and a second material being calcium sulphate,
b) contacting/incubating/soaking two or more pharmaceutical compounds with the material in a) wherein the two or more pharmaceutical compounds are;
I) vancomycin and rifampicin in combination, or
II) gentamicin and rifampicin,
c) separating non-bound pharmaceutical compounds from the material obtained in step b).

26. The method according to claim 25, wherein the hydroxyapatite in any configuration and the calcium sulphate in any configuration, crystal form or degree of hydration.

27. (canceled)

28. (canceled)

29. (canceled)

30. The method according to claim 25, wherein the one or more pharmaceutical compounds are dissolved in any suitable solvent such as a physiological saline solution, water or other aqueous solution, buffered solution, or any suitable organic solvent or any mixtures thereof, and thereafter contacted with the first and/or second material.

31. The method according to claim 25, wherein the period during which the one or more pharmaceutical compounds is contacted with the particulate first material and/or a second material, may be within range of minutes, hours or days, such as e.g. about 1 hour, such as e.g. about 3 hours, such as e.g. about 6 hours, such as e.g. about 12 hours, such as e.g. about 24 hours, such as e.g. about 48 hours etc.

32. The method according to claim 25, wherein after contacting/incubating the one or more pharmaceutical compounds with the particulate first material and/or a second material, the resulting mixture may be separated from the solution or suspension comprising the one or more pharmaceutical compounds by centrifugation or filtration etc. and wherein the resulting particulate material may be rinsed with any suitable solvent, and optionally thereafter allowed to dry.
